(19) United States
(12) Patent Application Publication
(10) Pub. No.: US 2023/0293478 A1
(43) Pub. Date: Sep. 21, 2023

(54) NITROGEN-CONTAINING ANALOGS OF SALINOMYCIN FOR USE IN ACUTE MYELOID LEUKEMIA

(71) Applicants: Centre National de la Recherche Scientifique (CNRS), Paris (FR); Institut National de la Santé et de la Recherche Médicale (INSERM), Paris (FR); Institut Curie, Paris (FR); Peter MacCallum Cancer Institute, Melbourne (AU)

(72) Inventors: Raphaël RODRIGUEZ, Ivry Sur Seine (FR); Mark DAWSON, Melbourne (AU); Sylvain GARCIAZ, Marseille (FR); Tatiana CANEQUE COBO, Paris (FR)

(73) Assignees: Centre National de la Recherche Scientifique (CNRS), Paris (FR); Institut National de la Santé et de la Recherche Médicale (INSERM), Paris (FR); Institut Curie, Paris (FR); Peter MacCallum Cancer Institute, Melbourne (AU)

(21) Appl. No.: 18/013,729

(22) PCT Filed: May 12, 2021
(86) PCT No.: PCT/EP2021/062729

§ 371 Date: Dec. 29, 2022

(30) Foreign Application Priority Data
Jul. 3, 2020 (EP) 20305750.0

(51) Int. Cl. A61K 31/35; A61K 31/635; A61K 45/06; A61P 35/02

(52) U.S. Cl. CPC A61K 31/35; A61K 31/635; A61K 45/06; A61P 35/02

(57) ABSTRACT

The invention relates compound of formula (I), enantiomers, mixture of enantiomers, diastereoisomers and mixture of diastereoisomers thereof: wherein W, X, Y and Z are as defined, for use in the treatment of Acute Myeloid Leukemia (AML).

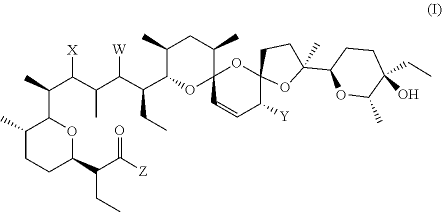

### FIELD OF THE INVENTION

The present invention relates to the field of treatment of subject affected by an Acute Myeloid Leukemia (AML), as well as associated therapeutic uses and methods.

### BACKGROUND OF THE INVENTION

Acute myeloid leukemia (AML) is a clinically and biologically heterogeneous clonal disease characterized by the accumulation of immature transformed progenitors in bone marrow.

For the past three decades standard intensive induction therapy (first-line therapy) is based on a combination of cytarabine plus anthracycline cytotoxic chemotherapy. Despite a high rate (70-80%) of complete remission after standard front-line chemotherapy, the prognosis remains poor, especially for older patients. Indeed, most patients will relapse after achieving complete remission, and treatment of refractory and relapsed AML remains a challenging issue.

Therefore, new therapeutic approaches are urgently needed.

Mitochondria are intracellular organelles performing many key roles in the cell, most notably oxidative phosphorylation (OxPhos), central carbon metabolism and the biosynthesis of intermediates for cell growth. Although it has been described a long time ago that cancer cells rely mainly on aerobic glycolysis to proliferate, recent publications have underlined that metabolic vulnerabilities of leukemic cells are in fact related to mitochondrial metabolism (Baccelli et al., 2019). In particular, leukemic stem cells as well as chemotherapy-resistant leukemic cells have been found to develop OxPhos dependency, raising the prospect that therapeutic strategies targeting mitochondria would be of interest (Dobson et al., 2020).

The inventors discovered that Ironomycin, an alkyne derivative initially named AM5, that is ten times more potent that the parental Salinomycin (Mai et al., 2017), takes advantage of cellular respiration to kill AML cells, inducing a remodeling of AML metabolism and a mitochondrial ATF4-dependent stress. Mechanistically, sequestrations of iron into the lysosome by the Ironomycin induces an imbalance in mitochondrial iron content, produces mitochondrial ROS and disorganizes cristae structure leading to nonapoptotic cell death. This new modality of mitochondrial dependent non-apoptotic cell death was found to be highly synergistic with BH3 mimetics such as the Bcl2 inhibitor venetoclax in vitro. These findings uncovered a complex interaction between metabolism regulation and cell death and raise the prospect that ironomycin may be useful for patients with AML.

### SUMMARY OF THE INVENTION

A first object of the present invention is a compound of formula (I), enantiomers, mixture of enantiomers, diastereoisomers and mixture of diastereoisomers thereof:

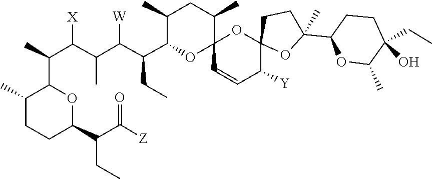

wherein:
W is selected from the group consisting of ═O; —NR₁R₂; —NR₃—(CH₂)ₙ—NR₄R₅; —O—(CH₂)ₙ—NR₄R₅;
NR₃—(CH₂)ₙ—N⁺R₆R₇R₈ and —O—(CH₂)ₙ—N⁺R₆R₇R₈;
X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
   R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
   R₃ is selected from the group consisting of H; (C₁-C₆)-alkyl; (C₁-C₆)-alkyl-aryl;
   R₄ and R₅, identical or different, are selected from the group consisting of H; (C₁-C₆)-alkyl; aryl and (C₁-C₆)-alkyl-aryl;
   R₆, R₇ and R₈, identical or different, are selected from the group consisting of (C₁-C₆)-alkyl; aryl and (C₁-C₆)-alkyl-aryl;
Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
   R₉ and R₁₀, identical or different, are selected from the group consisting of H, (C₁-C₆)-alkyl, aryl and (C₁-C₆)-alkyl-aryl;
   R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
   R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl
n=0, 2, 3, 4, 5 or 6,
   with the proviso that at least one of W, X and Y is selected from the group consisting of —NR₁R₂; —NR₃—(CH₂)ₙ—NR₄R₅; —O—(cH₂)ₙ—NR₄R₅; —NR₃—(CH₂)ₙ—N⁺R₆R₇R₈ and —O—(CH₂)ₙ—N⁺R₆R₇R₈,
   for use in the treatment of Acute Myeloid Leukemia (AML).

The invention also relates to a pharmaceutical composition comprising, in a pharmaceutical acceptable vehicle, the compounds of the invention of formula (I), enantiomers, mixture of enantiomers, diastereoisomers and mixture of diastereoisomers thereof, for use in a method for treating subjects having Acute Myeloid Leukemia (AML).

In a particular embodiment, the pharmaceutical composition is for use in a method for treating unfit subjects, in particular older subjects.

In another particular embodiment, the pharmaceutical composition is for use in a method for treating subjects likely to display an AML relapse and/or death, or subjects refractory or resistant to a first line treatment. Said subjects are also named subjects with poor outcome.

Another subject-matter of the invention is a pharmaceutical product comprising:
(i) a compound of formula (I) of the present invention and
(ii) another anti-cancer agent selected from the group consisting of agents used in chemotherapy, targeted treatments, immune therapies, and combinations thereof,
   as combination product for simultaneous, separate or staggered use as a medicament in the treatment of AML.

In a particular embodiment, the pharmaceutical product comprises
(i) a compound of formula (I) of the invention wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in chemotherapy such as anthracyclin, aracytine, azacitidine, or an agent used in targeted treatments such as Bcl2 inhibitor, Mcl1 inhibitor or other BH3 mimetics, FLT3 inhibitors, IDH1/IDH2 inhibitors, or a combination thereof, preferably a Bcl-2 inhibitor or other BH3 mimetic.

In a preferred embodiment, the pharmaceutical product comprises
(i) a compound of formula (I) of the invention wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in targeted treatments such as Bcl2 inhibitor, Mcl1 inhibitor or other BH3 mimetics, FLT3 inhibitors, IDH1/IDH2 inhibitors, or a combination thereof, preferably Bcl-2 inhibitor or other BH3 mimetic.

In a particular embodiment, the pharmaceutical product may comprise:
(i) a compound of formula (I) of the invention wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl,
(ii) an agent used in targeted treatments such as Bcl2 inhibitor, Mcl1 inhibitor or other BH3 mimetics, FLT3 inhibitors, IDH1/IDH2 inhibitors, or a combination thereof, preferably Bcl-2 inhibitor or other BH3 mimetic, and
(iii) an additional agent used in chemotherapy such as anthracyclin, aracytine, azacytidine.

The present invention also relates to the pharmaceutical product according to the invention for use in a method for treating unfit subjects, in particular older subjects, or for use in a method for treating subjects likely to display an AML relapse and/or death, or subjects refractory or resistant to a first-line treatment.

Another object of the present invention is a compound targeting iron metabolism and disturbing intra-mitochondrial iron equilibrium, for use in the treatment of Acute Myloid Leukemia (AML).

### Definitions

Salinomycin is a monocarboxylic polyether possessing ionophoric properties of the following formula:

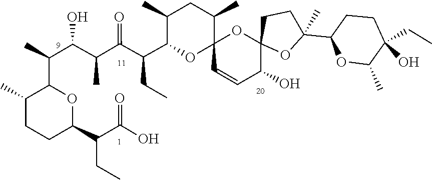 [SMILES: CCC(C(=O)O)[C@H]1CC[C@H](C)C([C@@H](C)[C@H](O)[C@H](C)C(=O)[C@H](CC)[C@H]2O[C@]3(C=C[C@@H](O)[C@]4(CC[C@@](C)([C@H]5CC[C@](O)(CC)[C@H](C)O5)O4)O3)[C@H](C)C[C@@H]2C)O1]

and the compounds used according to the present invention in Acute Myeloid Leukemia (AML) treatment are 9- and/or 11- and/or 20-amino derivatives of salinomycine, in particular 20-amino derivatives of salinomycine as disclosed in the patent application WO2016/038223. These derivatives of salinomycin are synthetic small molecules chemically derived from salinomycin exhibiting a more potent activity and potentially lower toxicity against healthy cells.

The term ‘subject’ or ‘patient’ or ‘individual’ refers to a human subject, whatever its age or sex. The subject is affected by an Acute Myeloid Leukemia (AML). The subject may be already subjected to a treatment, by any chemotherapeutic agent, or may be untreated yet.

The term ‘AML subject’ refers to a subject having AML originating from a population of AML subjects, from early to late stage of AML, the said subjects undergoing or not undergoing a therapeutic treatment.

In a particular embodiment, the AML subject is an unfit subject.

In another particular embodiment, the AML subject is likely to display an AML relapse and/or death, or is refractory or resistant to a first line treatment. such AML subject is also named as having a ‘poor outcome’ or ‘poor prognosis’. In a particular embodiment, the AML subject has recurrent known genetic abnormalities with a non-favorable cytogenetic risk. In another embodiment, the AML subject is likely to display an AML relapse.

By ‘unfit subject’ or ‘unfit patient’, it means a subject unfit for standard treatment, in particular among older adults, generally based on their performance status, physical function, and comorbidity. These patients may have subclinical impairments that limit resilience when stressed with intensive therapies.

By ‘first-line treatment’ or ‘first-line therapy’, it means treatment regimen or regimens that are generally accepted by the medical establishment for initial treatment of a given type and stage of cancer. Second-line therapies are those tried when the first ones do not work adequately, ie have some limited efficacy, or produce unacceptable side effects, damage organs in the body.

The term “treating” or “treatment” means stabilizing, alleviating, curing, or reducing the progression of the AML.

By ‘compound targeting iron metabolism’ according to the invention, it means iron chelators and small molecules sequestering iron into the lysosome and inducing an imbalance in mitochondrial iron content. Iron chelators are small molecules susceptible to interact reversibly with iron. And small molecules sequestering iron into lysosome are loose iron binders that accumulate in the lysosome able to block the metal in this organelle and induce an imbalance in mitochondrial iron content.

### BRIEF DESCRIPTION OF THE FIGURES

FIG. 1: Resistance Crispr screen identifies PGP and HK2 as two major regulators of ironomycin resistance. a (upp), Proliferation curves of MV4; 11, MOLM-13 and OCI-AML3 cell lines treated with ironomycin at indicated doses. a (down) IC50 of AML cell lines after 72 h of treatment with ironomycin. Cell death was assessed with flow cytometry using propidium iodide (PI) fluorescence on three biological replicates. Flow cytometry analysis of propidium iodide (PI) fluorescence in OCI-AML3, MOML13 and MV4; 11 cell lines treated with ironomycin at indicated time and doses. Data are normalized on DMSO and means±SD of three biological replicates are shown. b. Immunoblot showing protein expression of PGP and HK2 in MV4; 11 expressing Cas9 transfected with a non-targeted sgRNA (NT) and two independent sgRNAs targeting either PGP or HK2. c-d. Competition assays validating two independent KO cell lines transfected either with HK2 or PGP shRNAs or a non-targeted (NT) sgRNA. Cells were treated with DMSO (c) or ironomycin at 500 nM (d) for the indicated time. Data are means±SD of two biological replicates for each KO cell lines.

FIG. 1 (continued): HK2 and PGP are modulators of ironomycin sensitivity. e, Immunoblot showing protein expression of PGP and HK2 in MV4; 11 expressing Cas9 and MV4; 11 transfected with a pool of three sgRNAs targeting either PGP or HK2. Cells were treated with ironomycin for the indicated doses for 24 hours. f-g, Competition assays using cell lines transfected with a pool of three sgRNAs targeting either HK2 or PGP. Cells were treated with 100 nM (f) or 500 nM ironomycin (g) for the indicated time. Data are normalized on DMSO and standardized on Day-0. Means±SD of three biological replicates are shown. h, Competition assays using cell lines transfected with a pool of three sgRNAs targeting either HK2 or PGP. Cells are treated with 50 μM AM23 for the indicated time. One biological replicate is shown. i, Competition assays using cell lines transfected with a pool of three sgRNAs targeting either HK2 or PGP. Cells were treated with 50 nM venetoclax for the indicated time. Means±SD of two biological replicates are shown.

FIG. 2: Metabolic plasticity impacts on ironomycin sensitivity. a. Schematic representation of glycolysis and the branched pentose phosphate pathway, with knocked out enzymes (HK2) and (PGP) b. Flow cytometry analysis of propidium iodide (PI) fluorescence in WT MV4; 11 cell line treated with 500 nM ironomycin for 48 h in RPMI medium with different concentration of glucose. Data are means±SD of three biological replicates. c Flow cytometry analysis of propidium iodide (PI) fluorescence in MOLM-13. Cells were pretreated with the pentose phosphate pathway inhibitor 6-aminonicotinamide (6AN, 10 μM) for 30 minutes and treated with 500 nM ironomycin for 48 h. Data are means ±SD of three biological replicates. d. Flow cytometry analysis of PI fluorescence in MV4; 11 cell line treated with ironomycin as indicated for 48 h with metformin (MET). Data are means±SD of two biological replicates.

FIG. 2 (continued): Biguanides modulate ironomycin sensitivity. e, Flow cytometry analysis of PI fluorescence in MV4; 11 cell line treated with ironomycin as indicated for 48 h with phenformin (Phen). Data are means±SD of two biological replicates. f, Combination index (CI) plot showing the CI for combined treatment with ironomycin+metformin (Met) (left) and ironomycin+Phen (right) in WT MV4; 11 cell line. The results of two biological replicates are shown. g,h, Flow cytometry analysis of PI fluorescence in PGP and HK2 KO cell lines treated with ironomycin in comparison with MV4; 11 transfected with a non-targeting sgRNA (NT). Cells were treated with ironomycin at the indicated time for 48 h without or with metformin (g) or phenformin (h). Data are means±SD of two biological replicates. i, Combination index (CI) plot showing the CI for combined treatment with ironomycin+metformin (Met) in PGP and HK2 KO cell lines. The results of two biological replicates are shown.

FIG. 3: Ironomycin remodels AML cell metabolism. a-e , Expression level of selected metabolites from the metabolomics analysis (mean values±SD from four biological replicates). Metabolites from glycolysis (a), tricarboxylic cycle acid (b), NADH (c) amino-acid (d) and nucleic acid metabolisms (e) are shown in bargraphs comparing DMSO conditions and 500 nM ironomycin treatment for 24 hours. The results of four biological replicates are shown (mean values±SD). *p<0.05, **p<0.01. ***p<0.001.

FIG. 4: Ironomycin induces a mitochondrial stress response. a, mRNA expression analysis of selected genes from the mitostress response pathway in MV4; 11 cells (left) and OCI-AML3 cells (right) 6 hours after exposure to ironomycin (500 nM). RT-QPCR of the selected genes was performed, using β2m as a housekeeping gene to normalize the data. Data from three technical replicates from one single representative experiment is shown b, mRNA expression analysis of selected genes from the mitostress response pathway in MV4; 11 cells treated with DMSO or ironomycin in normal and in high glucose conditions. RT-QPCR of the selected genes was performed, using β2m as a housekeeping gene to normalize the data. Data from three technical replicates from one single representative experiment is shown. c, Immunoblot showing protein expression of ATF4 after ironomycin (500 nM) at early time points.

FIG. 5: Ironomycin promotes iron-dependent mitochondrial oxidative stress. a, Flow cytometry analysis of propidium iodide (PI) fluorescence in MOLM13 cell line. Cells were pretreated with the ferroptosis inhibitor ferrostatin (ferro, 20 μM) or liproxstatin (lipro, 10 μM) for 30 minutes and treated with ironomycin (irono, 500 nM) or RSL-3 for 24 hours. Data are means±SD of two biological replicates. b, Flow cytometry analysis of RhoNox-M positive MOLM13 cells. N=3 independent biological replicates c, ICP-MS measurements of cellular iron in MOLM13 cells. N=3 independent biological replicates. d, Flow cytometry analysis of mitochondrial ROS using Mitosox Red fluorescence dye in MV4; 11 and OCI-AML3. Cells were treated with ironomycin (irono, 500 nM) for 24 h. Data are means±SD of three biological replicates. e, Transmission electron microscopy images of mitochondria in MV4; 11 cells treated with or without 500 nM ironomycin for 16 h. Black arrows indicate cristae. Micrographs were taken at 12.500×. Scale bar, 500 nm.

FIG. 5 (continued): Ironomycin cell death is not canonical ferroptosis. f, Flow cytometry analysis of lipid ROS using C11 BODIPY 581/591 (BODIPY C11) from one representative experiment showing oxidized C11 after 48 hours of ironomycin treatment (500 nM). g, Flow cytometry Analysis of lipid ROS using C11 BODIPY 581/591 (BODIPY C11) in OCIAML3 and MOLM-13 cells treated with ironomycin (500 nM) for 48 hours. Each data point represents the ratio of oxidized (C11ox) to total non-oxidized C11 (C11Non-ox+C11ox) median fluorescent intensity (MFI) from four independent biological replicates. h, Flow cytometry analysis of propidium iodide (PI) fluorescence in OCI-AML3 cell line. Cells were pretreated with the ferroptosis inhibitor ferrostatin (Ferro) (20 μM) or liproxstatin-1 (Lipro, 10 μM) for 30 minutes and treated with ironomycin (irono) (500 nM) or RSL-3 (50 nM) for 24 h. Data are means±SD of two biological replicates. i, Analysis of lipid ROS using C11 BODIPY 581/591 (BODIPY C11) by flow cytometry in OCI-AML3 and in MOLM-13 cells treated with ironomycin (irono, 500 mM) for 48 hours or RSL3 (50 nM) for 6 hours. Cells were pretreated with the ferroptosis inhibitor Liproxstatin-1 (Lipro) (10 μM) for 30 minutes. Bargraphs represent the ratio of oxidized (C11ox) to total non-oxidized C11 (C11Non-ox+C11ox) median fluorescence intensity (MFI) from two independent biological replicates.

FIG. 5 (continued): Metformin antagonizes with ironomycin by counterbalancing mitochondrial iron content. j, Flow cytometry analysis of mitochondrial fe(II) in MOLM13 cell line using a turn-off mitochondrial Fe2+ probe. Cells were treated with metformin and ironomycin as indicated for 6 and 24 h. Bar chart represent an average of 3 independent biological replicates. k, Flow cytometry analysis of mitochondrial ROS using Mitosox Red fluorescence dye in MV4; 11 transfected with a HK2, PGP and non-targeting sgRNA (NT). Cells were treated with ironomycin (irono, 500 nM) for 24 h. Data are means±SD of three biological replicates.

FIG. 6: Ironomycin induces a mitochondrial-dependent cell death pathway and disturbs intraorganelle iron equilibrium. a, b, Cell death analyses of WT MV4; 11 cell line. Cell death was measured with PI staining by Flow Cytometry and Cell titer glow analysis after 24 h of venetoclax (a) or ironomycin (b) treatment at the indicated doses. The results of these two experiments are pooled together with mean values and SD. c, Immunoblot showing protein expression of cleaved PARP1 and cleaved caspase 3 (casp. 3) with ABT-199 Venetoclax (50 nM) or ironomycin (500 nM) for 24 h in MOLM-13 cell line. d, Flow cytometry analysis of Annexin V-FITC fluorescence in MOLM-13. Cells were pretreated with the pan caspase inhibitor Z-VAD-fmk (Z) (50 μM) for 30 minutes and treated with ironomycin (irono, 500 nM) or venetoclax (50 nM) for 24 h. Data are means±SD of three biological replicates. e, Flow cytometry analysis of propidium iodide (PI) fluorescence in MOLM-13. Cells were pretreated with the necroptosis inhibitor Nec:rostatin-1 (N) (10 μM) for 30 minutes and treated with ironomycin (500 nM) or Birinapant (Bir) (100 nM) and IDN-6556 (IDN, 5 μM) for 16 h. Data are means±SD of three biological replicates. f, Flow cytometry analysis of PI fluorescence in MV4; 11 cell line treated with ironomycin in association with venetoclax at the indicated doses for 48 h. Data are means±SEM of three biological replicates. g, Combination index (CI) plot showing the CI for combined treatment with ironomycin+venetoclax in WT MV4; 11 and OCI-AML3 cell lines. The results of three biological replicates are shown.

FIG. 7: Ironomycin shows marked synergy with BH3 mimetics and overcomes resistance to venetoclax. A, Heatmaps showing the percentage of inhibition assessed by FACS (PI) using ironomycin and venetoclax as single agent and in combination in two AML cell lines (top) and Bliss calculation measuring synergy between the two drugs (bottom). Data from three biological replicates are normalized on vehicle condition. B, Analysis of mitochondrial membrane potential (Δ ψm) using JC-1 staining assessed by flow cytometry after 24 hours of low dose venetoclax with or without low dose ironomycin treatments. Loss of JC-1 staining is associated with a loss of Δ ψm. Data show mean±SEM of 3 independent experiments. C, Immunoblot showing cleaved caspase 3 after 24 hours of low dose venetoclax with or without low dose ironomycin treatments in OCI-AML3 cell line treated with ironomycin and venetoclax. D, Transmission electron microscopy images of mitochondria in MV4; 11 WT treated with low doses of ironomycin and venetoclax for 36 hours. Black arrowheads—disrupted mitochondrial integrity. Scale bars, 2 μm top, 500 nm bottom panels. E, Kaplan-Meyer analyses showing survival of NSG mice transplanted with MV4,11 cells (5 mice per cohorts) treated with ironomycin 1 mg/kg by IP (5 days/week for 4 weeks), venetoclax 75 mg/kg by oral gavage (5 days/week for 4 weeks) and combination of the two drugs (* and ** indicates p<0.05 and p<0.01 respectively). F, Heatmaps showing the effect on cell viability assessed by flow cytometry (PI) in response to escalating doses of ironomycin and venetoclax for 5 days as single agent and in combination in five patients known to be clinically resistant to venetoclax (top) and Bliss calculation measuring synergy between the two drugs (bottom). Data is indicative of one experiment and are normalized on the DMSO control.

FIG. 8: Combination of ironomycin and venetoclax tolerance in NSG mice. Tolerance of the venetoclax plus ironomycin combination (ven-irono) in NSG mice. Curves show white cell counts [WCC, (A)], Hb level [Hb, (B)], platelet count [PLT, (C)] and body weight [BW, (D)], in 5 treated with ven-irono and 5 control mice treated with vehicle. Doses of treatment were combination of ironomycin 1 mg/kg by IP (5 days/week for 4 weeks), venetoclax 75 mg/kg by oral gavage (5 days/week for 4 weeks).

FIG. 9: Ironomycin mechanism of action. ironomycin induces a non-apoptotic mitochondrial cell death dependent on cellular respiration. Molecular mechanism involves iron sequestration into the lysosomes, mitochondrial iron imbalance leading to cristae disorganization, oxidative stress and a nonapoptotic cell death.

### DETAILED DESCRIPTION OF EMBODIMENTS OF THE INVENTION

The inventors have demonstrated in the examples later in the description that a compound of formula (I) according to the invention, Ironomycin, takes advantage of cellular respiration to kill AML cells, inducing a remodeling of AML metabolism and a mitochondrial ATF4-dependent stress.

So the present invention concerns a compound of formula (I), enantiomers, mixture of enantiomers, diastereoisomers and mixture of diastereoisomers thereof:

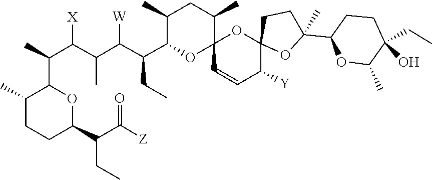

wherein:
W is selected from the group consisting of ═O; —NR₁R₂; —NR₃—(CH₂)ₙ—NR₄R₅;
NR₃—(CH₂)ₙ—N⁺R₆R₇R₈ and —O—(CH₂)ₙ—N⁺R₆R₇R₈;
X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
   R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
   R₃ is selected from the group consisting of H; (C₁-C₆)-alkyl; (C₁-C₆)-alkyl-aryl;
   R₄ and R₅, identical or different, are selected from the group consisting of H; (C₁-C₆)-alkyl; aryl and (C₁-C₆)-alkyl-aryl;
   R₆, R₇ and R₈, identical or different, are selected from the group consisting of (C₁-C₆)-alkyl; aryl and (C₁-C₆)-alkyl-aryl;
Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
   R₉ and R₁₀, identical or different, are selected from the group consisting of H, (C₁-C₆)-alkyl, aryl and (C₁-C₆)-alkyl-aryl;
   R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
   R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl
n=0, 2, 3, 4, 5 or 6,
   with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
   for use in the treatment of Acute Myeloid Leukemia (AML).

In the sense of the present invention,:
“(C₁-C₁₆)-alkyl” designates an optionally substituted acyclic, saturated, linear or branched hydrocarbon chain comprising 1 to 16 carbon atoms. Examples of (C₁-C₁₆)-alkyl groups include methyl, ethyl, propyl, butyl, pentyl, hexyl, heptyl, octyl, and dodecyl;
“—(C₃-C₁₆)-alkenyl” designates an optionally substituted acyclic, saturated, linear or branched hydrocarbon chain comprising 3 to 16 carbon atoms, at least two of which are linked via a double bond. Examples of “—(C₃-C₁₆)-alkenyl” include propenyl, butenyl, pentenyl or hexenyl;
“C₃-C₁₆)-alkynyl” designates an optionally substituted acyclic, saturated, linear or branched hydrocarbon chain comprising 3 to 16 carbon atoms, at least two of which are linked via a triple bond. Examples of “—(C₃-C₁₆)-alkynyl” include propynyl, butynyl, pentynyl or hexynyl;
“(C₃-C₁₆)-cycloalkyl” designates an optionally substituted cyclic, saturated hydrocarbon chain comprising 1 to 16 carbon atoms. Examples of (C₃-C₁₆)-cycloalkyl groups include cyclopropyl, cyclobutyl, cyclopentyl, cyclohexyl, cycloheptyl, cyclooctyl, and cyclododecyl. Advantageously, (C₃-C₁₆)-cycloalkyl group is selected from optionally substituted cyclopropyl, cyclobutyl and cyclopentyl;
“aryl” designates an aromatic, monocyclic ring that may be fused with a second saturated, unsaturated or aromatic ring. The term aryl include, without restriction to the following examples, phenyl, indanyl, indenyl, naphtyil, anthracenyl, phenanthrenyl, tetrahydronaphtyl, and dihydronaphtyl. The preferred aryl are those comprising one six-membered aromatic ring. The aryl group may be substituted with one or more groups independently selected from the group consisting of alkyl, alkoxy, halogen, hydroxyl, amino, nitro, cyano, trifluoro, carboxylic acid or carboxylic ester. Examples of substituted phenyl groups are methoxyphenyl, dimethoxyphenyl, trimethoxyphenyl, fluorophenyl and trifluoromethylphenyl;
“—(C₁-C₆)-alkyl-aryl” designates in the sense of the present invention an aryl group, as defined above, linked to the rest of the molecule by an alkyl chain containing 1 to 6 carbon atoms. Advantageously, the —(C₁-C₆)-alkyl-aryl is a substituted or unsubstituted benzyl.

Examples of substituted benzyl groups include hydroxybenzyl, methoxybenzyl, cyanobenzyl, nitrobenzyl or fluorobenzyl;
“heteroaryl” designates a mono- or polycyclic aryl as defined above where one or more carbon atoms have been replaced with one or more heteroatoms selected from the group consisting of N, O and S. Examples of heteroaryl groups include furyl, thienyl, imidazolyl, pyridyl, pyrrolyl, N-alkyl pyrrolyl, pyrimidinyl, pyrazinyl, tetrazolyl, triazolyl and triazinyl. The heteroaryl group may be substituted with one or more groups independently selected from the group consisting of alkyl, alkoxy, halogen, hydroxyl, amino, nitro, cyano, trifluoro, carboxylic acid or carboxylic ester. Preferred heteroaryls are those having 5 or 6 atoms in the ring, such as indolyl, pyrrolyl, pyridinyl, pyrrazolyl, triazolyl, furanyl or thienyl.
“—(C₁-C₆)-alkyl-heteroaryl” designates in the sense of the present invention an heteroaryl group, as defined above, linked to the rest of the molecule by an alkyl chain containing 1 to 6 carbon atoms. Advantageously, the “—(C₁-C₆)-alkyl-heteroaryl” is a substituted or (C₁)-alkyl-heteroaryl.

The term “optionally substituted” as used herein means that any of the hydrogen atoms can be replaced by a substituent selected from the group consisting of alkyl, alkoxy, halogen, hydroxyl, amino, nitro, cyano, trifluoro, carboxylic acid or carboxylic ester

Advantageously, R₁ and R₂, identical or different, are selected from the group consisting of H; (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₅)-cycloalkyl; (C₁-C₆)-alkyl-aryl, advantageously benzyl, and (C₁-C₆)-alkyl-heteroaryl, advantageously CH₂-pyridynyl.

Advantageously, R₁ and R₂ are not both H.

More advantageously, R₁ is H and R₂ is selected from the group consisting of (C₁-C₁₆)-alkyl, advantageously (C₃-C₁₄)-alkyl, more advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₃ is selected from the group consisting of H and (C₁-C₆)-alkyl. Preferably, R₃ is H.

Advantageously, R₄ and R₅, identical or different, are selected from the group consisting of H and (C₁-C₁₆)-alkyl. More advantageously, R₄ and R₅ are H or (C₁-C₆)-alkyl. Preferably, R₄ and R₅ are identical. In one advantageous embodiment, the group —(CH₂)ₙ—NR₄R₅ is selected from the group consisting of —(CH₂)₂—N(CH₃)₂, —(CH₂)₃—N(CH₃)₂, —(CH₂)₂—NH₂ and —(CH₂)₃—NH₂.

Advantageously, R₅, R₇ and R₈, identical or different, are selected from the group consisting of (C₁-C₆)-alkyl; and aryl. More advantageously, R₆, R₇ and R₈ are (C₁-C₆)-alkyl. Preferably, R₆, R₇ and R₈ are identical. In one advantageous embodiment, the group —(CH₂)ₙ—N⁺R₆R₇R₈ is selected from the group consisting of —(CH₂)₂—N⁺(CH₃)₃, and —(CH₂)₃—N⁺CH₃)₃.

Advantageously, Z is OH, OOH, NHNH₂, NHOH, or NH₂OH, preferably OH.

In an advantageous embodiment according to the present invention, the compound of formula (I) is a monoamine derivative of salinomycine, and only one of W, X or Y is a —NR₁R₂; —NR₃—(CH₂)ₙ—NR₄NR₅; —O—(CH₂)ₙ—NR₄R₅; —NR₃—(CH₂)ₙ—N⁺R₆R₇R₈; or —O—(CH₂)ₙ—N⁺R₆R₇R₈ group, and W, X, Y, Z, R₁, R₂, R₃, R₄, R₅, R₆, R₇, R₈ and n are as defined in formula (I).

The compound of formula (I) used in the present invention is advantageously a 20-amino derivative of salinomycine of formula (Ic3) as disclosed in WO2016/038223:

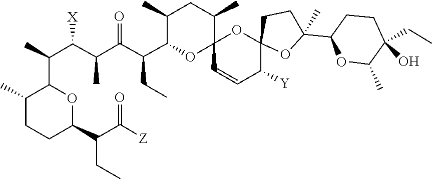

wherein:

X is selected from the group consisting of OH and ═O,

Y is selected from the group consisting of —NR₁NR₂; —NR₃—(CH₂)ₙ—NR₄R₅; —O—(CH₂)ₙ—NR₄R₅; —NR₃—(CH₂)ₙ—N⁺R₆R₇R₈; and —O—(CH₂)ₙ—N⁺R₆R₇R₈; and

Z, R₁, R₂, R₃, R₄, R₅, R₆, R₇, R₈ and n are as defined in formula (I).

In one advantageous embodiment, in the compounds of formula (Ic3), X is OH, Z is OH, and

Y is —NR₁R₂. More advantageously, R₁ is H and R₂ is selected from the group consisting of (C₁-C₁₆)-alkyl, advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

In another embodiment, X is ═O, Y is selected from the group consisting of ═N—OH and NR₁R₂ and Z is NHOH. Advantageously, X is ═O, Y is NR₁R₂ and Z is NHOH. More advantageously, R₁ is H and R₂ is CH₂-pyridinyl, preferably CH₂-(2-pyridinyl). Alternatively, R₁ is H and R₂ is (C₃-C₁₆)-cycloalkyl or (C₃-C₁₆)-alkynyl.

In a particular embodiment, when Z is —NHOH, W is ═O and X is —OH, then Y is not a propargyl group.

In a particular embodiment, when Z is —OH, W is ═O and X is —OH, then Y is not NCH₂CH₂N(CH₃)₂.

The compounds of formula (I) used according to the invention, and methods of synthesis of said compounds are disclosed in the patent application WO2016/038223.

In a particular and preferred embodiment, the compound of formula (I) is as defined above, wherein X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

In a more preferred embodiment, the compound of formula (I) is as defined above, wherein X is OH, Z is OH and Y is NR₁R₂ where R₁ is H and R₂ is selected from the group consisting of (C₈-C₁₄)-alkyl; (C₃-C₅)-alkenyl; (C₃-C₅)-alkynyl, (C₃-C₆)-cycloalkyl, benzyl, and CH₂-pyridynyl, preferably (C₃-C₅)-alkynyl.

In a particular embodiment, W is ═O, X is —OH, Y is —NR₁R₂ preferably with R₁ being H and R₂ being (C₃-C₁₆)-alkynyl, preferably propargyl, and Z is —OH. Such compound is also named Ironomycin or compound AM5 as disclosed in the patent application WO2016/038223.

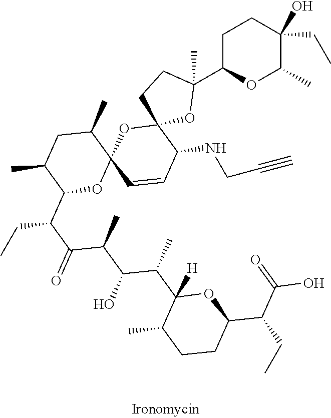 [SMILES: C#CCN[C@@H]1C=C[C@]2(O[C@H]([C@@H](CC)C(=O)[C@@H](C)[C@@H](O)[C@H](C)[C@@H]3O[C@@H]([C@@H](CC)C(=O)O)CC[C@@H]3C)[C@@H](C)C[C@H]2C)O[C@@]12CC[C@@](C)([C@H]1CC[C@](O)(CC)[C@H](C)O1)O2]

In a particular embodiment, W is ═O, X is —OH, Y is —NR₁R₂ preferably with R₁ being H and R₂ being (C₃-C₁₆)-cycloalkyl, preferably cyclopropyl, and Z is —OH. Such compound is also named AM23 as disclosed in the patent application WO2016/038223.

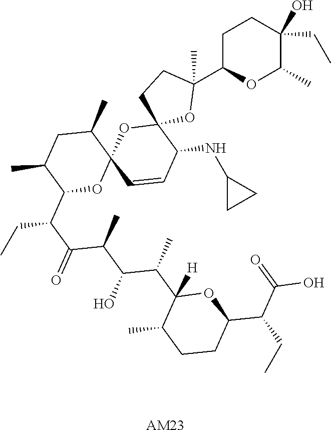

In another particular embodiment, W is ═O, X is OH, Z is OH, and Y is NR₁R₂ where R₁ is H and R₂ is a (C₃-C₆)-cycloalkyl group, in particular a substituted cyclopropyl as disclosed hereunder:

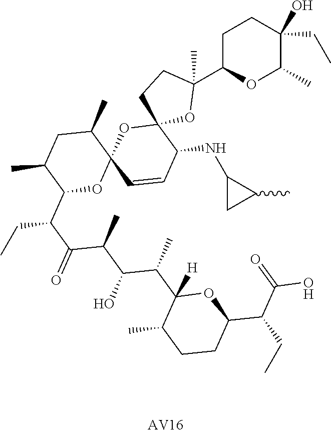 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NC3CC3C)[C@]3(CC[C@@](C)([C@H]4CC[C@](O)(CC)[C@H](C)O4)O3)O2)[C@H](C)C[C@@H]1C ; "AV16"]

In another particular embodiment, W is ═O, X is OH, Z is OH, and Y is NR₁R₂ where R₁ is H and R₂ is a (C₁-C₆)-alkyl-aryl group, in particular a benzyl group substituted by an hydroxy, as disclosed hereunder:

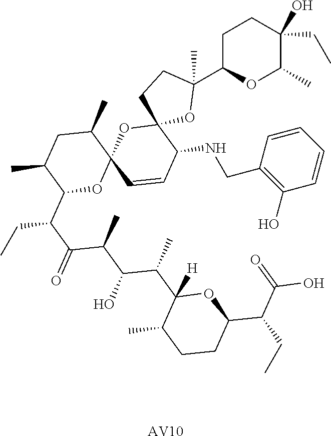 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3ccccc3O)[C@]3(CC[C@@](C)([C@H]4CC[C@](O)(CC)[C@H](C)O4)O3)O2)[C@H](C)C[C@@H]1C ; "AV10"]

In another particular embodiment, W is ═O, X is OH, Z is OH, and Y is NR₁R₂ where R₁ is H and R₂ is a (C₁-C₆)-alkyl-pyridyl group, in particular a CH₂-pyridinyl group, as disclosed hereunder:

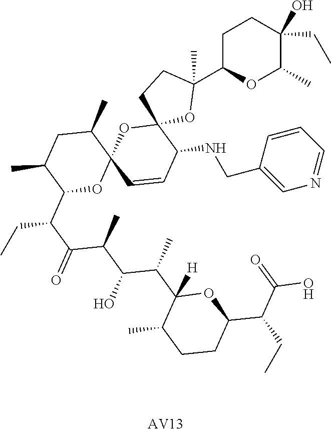 [SMILES: CC[C@@H](C(=O)[C@@H](C)[C@@H](O)[C@H](C)[C@@H]1O[C@@H]([C@@H](CC)C(=O)O)CC[C@@H]1C)[C@H]1O[C@]2(C=C[C@@H](NCc3cccnc3)[C@]3(CC[C@@](C)([C@H]4CC[C@](O)(CC)[C@H](C)O4)O3)O2)[C@H](C)C[C@@H]1C ; "AV13"]

The compounds AM5, AM23, AV10, AV13 and AV16, preferably AM5 and AM23 are particular and preferred compounds used in the pharmaceutical composition, pharmaceutical product and therapeutic uses disclosed hereunder.

### Pharmaceutical Composition

Another object of the invention is a pharmaceutical composition comprising in a pharmaceutical acceptable vehicle, at least a compound of formula (I) as disclosed above, for use in a method for treating subjects having Acute Myeloid Leukemia (AML).

In a particular embodiment, the compound of formula (I) is with W is ═O, X is OH, Z is OH, and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

In a preferred embodiment, the compound of formula (I) is with W is ═O, X is OH, Z is OH, and Y is NR₁R₂ where R₁ is H and R₂ is selected from the group consisting of (C₈-C₁₄)-alkyl; (C₃-C₅)-alkenyl; (C₃-C₅)-alkynyl, (C₃-C₆)-cycloalkyl, benzyl, and CH₂-pyridynyl, preferably (C₃-C₅)-alkynyl.

In a more preferred embodiment, the compound of formula (I) is with W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl, and most preferably propargyl.

The pharmaceutical composition for use according to the invention comprises at least one compound of formula (I) as defined above, a pharmaceutical salt, solvate or hydrate thereof, and at least one pharmaceutically acceptable excipient.

For the purpose of the invention, the term ‘pharmaceutically acceptable’ is intended to mean what is useful to the preparation of a pharmaceutical composition, and what is generally safe and non-toxic, for a pharmaceutical use.

The term «pharmaceutically acceptable salt, hydrate of solvate» is intended to mean, in the present invention, a salt of a compound which is pharmaceutically acceptable, as defined above, and which possesses the pharmacological activity of the corresponding compound.

Such salts comprise:
hydrates and solvates,
acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphtalenesulfonic, propionic, succinic, dibenzoyl-L-tartaric, tartaric, p-toluenesulfonic, trimethylacetic, and trifluoroacetic acid and the like, and
salts formed when an acid proton present in the compound is either replaced by a metal ion, such as an alkali metal ion, an alkaline-earth metal ion, or an aluminium ion;

or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The pharmaceutical compositions for use according to the invention can be intended to oral, sublingual, subcutaneous, intramuscular, intravenous, transdermal, topical or rectal administration. The active ingredient can be administered in unit forms for administration, mixed with conventional pharmaceutical carriers, to animals or to humans. When a solid composition is prepared in the form of tablets, the main active ingredient is mixed with a pharmaceutical vehicle and other conventional excipients known to those skilled in the art.

The compounds of the invention can be used in a pharmaceutical composition at a dose ranging from 0.01 mg to 1000 mg a day, administered in only one dose once a day or in several doses along the day, for example twice a day. The daily administered dose is advantageously comprised between 5 mg and 500 mg, and more advantageously between 10 mg and 200 mg. However, it can be necessary to use doses out of these ranges, which could be noticed by the person skilled in the art.

In a particular embodiment, the said pharmaceutical composition is used in a method for treating unfit subjects, in particular older subjects.

In another particular embodiment, the said pharmaceutical composition is used in a method for treating subjects likely to display an AML relapse and/or death, or subjects refractory or resistant to a first line treatment.

### Pharmaceutical Product (Also Named “Combination Product”)

The present invention also relates to a pharmaceutical product or combination product comprising:
(i) a molecule targeting iron metabolism, in particular an iron chelator or a small molecule sequestering mitochondrial iron and
(ii) another anti-cancer agent selected from the group consisting of agents used either in chemotherapy, in targeted treatments, in immune therapies, and in combinations thereof,
   as combination product for simultaneous, separate or staggered use as a medicament in the treatment of AML, in particular in AML unfit subjects or subjects having a poor outcome.

The expression “combination product” according to the invention means herein that the compound of formula (I) used in the present invention is administered to the subject treated before, during (including concurrently with-preferably co-formulated with) and/or after treatment of the subject with the other anti-cancer drug. The formulations may conveniently be presented in unit dosage form by methods known to those skilled in the art. Preferably, the kit-of-parts contains instructions indicating the use of the dosage form to achieve a desirable affect and the amount of dosage form to be taken over a specified time period.

In a particular embodiment, the present invention relates to a pharmaceutical product comprising:
(i) a compound of formula (I) according to the invention and
(ii) another anti-cancer agent selected from the group consisting of agents used either in chemotherapy, in targeted treatments, in immune therapies, and in combinations thereof,
   as combination product for simultaneous, separate or staggered use as a medicament in the treatment of AML, in particular in AML unfit subjects or subjects having a poor outcome.

By ‘agents used in chemotherapy’ according to the invention, it means drugs also named themo drugs' able to stop the growth of cancer cells, either by killing the cells or by stopping them from dividing.

In some embodiments, agents used in chemotherapy for AML treatment may include anthracyclin (daunorubicin, idarubicin), aracytine, and/or hypomethylating agents (azacytidine, decitabine).

By ‘agents used in targeted treatments’ according to the invention, it means drugs or other substances able to identify and attack specific types of cancer cells with less harm to normal cells. Some targeted therapies block the action of certain enzymes, proteins, or other molecules involved in the growth and spread of cancer cells. Other types of targeted therapies help the immune system kill cancer cells or deliver toxic substances directly to cancer cells and kill them. Targeted therapy may have fewer side effects than other types of cancer treatment. Most targeted therapies are either small molecule drugs or monoclonal antibodies. In some embodiments, agents used in targeted treatment for AML may include Bcl2 inhibitors (venetoclax), Mcl1 inhibitprs or other BH3 mimetics, IDH1/2 inhibitors (ivosidenib, enasidenib), and/or FLT3 inhibitors (midaustorine, gilteritinib . . . ).

By ‘agents used in immune therapies’ according to the invention, it means substances also named ‘immunomodulatory agents’ able to stimulate or suppress the immune system to help the body fight cancer. Some types of immunotherapy only target certain cells of the immune system. Others affect the immune system in a general way. Types of immunotherapy include as examples cytokines, and some monoclonal antibodies.

In some embodiments, agents used in immune therapies for AML may include mylotarg (gemtuzumab ozogamycin).

So in a particular embodiment for AML treatment, the molecule targeting iron metabolism (i) is a compound of formula (I) as defined above, wherein W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and the other compound (ii) is selected in the group consisting of agents used in chemotherapy such as anthracyclin, aracytine, azacitidine, agents used in targeted treatments such as Bcl2 inhibitor, Mcl1 inhibitor or other BH3 mimetics, FLT3 inhibitors, IDH1/IDH2 inhibitors, and in combinations thereof,

In a particular and preferred embodiment, the invention relates to a pharmaceutical product comprising:

(i) a compound of formula (I) as disclosed above wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) a agent used in chemotherapy such as anthracyclin, aracytine, azacitidine, or an agent used in targeted treatments such as Bcl2 inhibitor, Mcl1 inhibitor or other BH3 mimetics, FLT3 inhibitors, IDH1/IDH2 inhibitors, or a combination thereof, preferably Bcl-2 inhibitor or other BH3 mimetic.

In a particular embodiment, the agent used in targeted treatments is selected in the group consisting of Bcl2 inhibitor, Mcl1 inhibitor or other BH3 mimetics, FLT3 inhibitors, IDH1/IDH2 inhibitors, or a combination thereof, preferably Bcl-2 inhibitor or other BH3 mimetic.

In a preferred embodiment, the invention relates to a pharmaceutical product comprising:
(i) a compound of formula (I) as disclosed above wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in targeted treatments such as Bcl2 inhibitor, Mcl1 inhibitor or other BH3 mimetic, FLT3 inhibitors, IDH1/IDH2 inhibitors, or a combination thereof, preferably Bcl-2 inhibitor or other BH3 mimetic.

In another preferred embodiment, the invention relates to a pharmaceutical product comprising:
(i) a compound of formula (I) as disclosed above wherein W is ═O, X is OH, Z is

OH and Y is N₁R₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl,
(ii) an agent used in targeted treatments such as Bcl2 inhibitor, Mcl1 inhibitor or other BH3 mimetic, FLT3 inhibitors, IDH1/IDH2 inhibitors, or a combination thereof, preferably Bcl-2 inhibitor or other BH3 mimetic, and
(iii) an agent used in chemotherapy such as anthracyclin, aracytine, azacytidine.

BCL2 homology domain 3 (BH3) mimetics are promising drugs for hematologic malignancies that trigger cell death by promoting the release of proapoptotic BCL2 family members from antiapoptotic proteins.

Bcl-2 (B-cell lymphoma 2), encoded in humans by the BCL2 gene is the founding member of the Bcl-2 family of regulator proteins that regulate cell death (apoptosis), by either inhibiting (anti-apoptotic) or inducing (pro-apoptotic) apoptosis. BcL-2 is localized to the outer membrane of mitochondria, where it plays an important role in promoting cellular survival and inhibiting the actions of pro-apoptotic proteins. The anti-apoptotic members of the BCL2 family, including BCL2L1 (also known as BCLXL), BCL2L2, MCL1, and BCL2-related protein A1 (BCL2A1), antagonize the activation of the pro-apoptotic members BCL2-associated X apoptosis regulator (BAX) and BCL2 antagonist/killer 1 (BAK1) that are essential for triggering the mitochondrial apoptotic pathway. The pro-apoptotic proteins in the BcL-2 family, including Bax and Bak, normally act on the mitochondrial membrane to promote permeabilization and release of cytochrome C and ROS, that are important signals in the apoptosis cascade. These pro-apoptotic proteins are in turn activated by BH3-only proteins, and are inhibited by the function of BCL-2 and its relative BCL-XI.

BH3 mimetics interact with anti-apoptotic BCL2 proteins in a competitive manner, eventually reactivating the death signal. In particular BH3 mimetics compounds encompasse BH3 mimetic compound targeting Bcl2 protein and BH3 mimetic compound targeting Mcl1 protein. Targeted and selective Bcl-2 inhibitors have been developed. Mention may be made of ABT-737 and navitoclax (ABT-263), and preferably Venetoclax (ABT-199) that is a highly selective inhibitor, which inhibits Bcl-2, but not Bcl-xL or Bcl-w.

Mcl-1 inhibitors have also been developed (AZD5991, AMG-176 and S64315 are in clinical trials).

So, in a preferred embodiment, the invention relates to a pharmaceutical product comprising:
(i) the compound of formula (I) with W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and
(ii) a Bcl2 inhibitor or other BH3 mimetic, preferably venetoclax.

So, in a preferred embodiment, the invention relates to a pharmaceutical product comprising:
(i) the compound of formula (I) with W is ═O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and
(ii) BH3 mimetic, preferably the compound ABT-199 (Venetoclax) as a Bcl2-inhibitor.

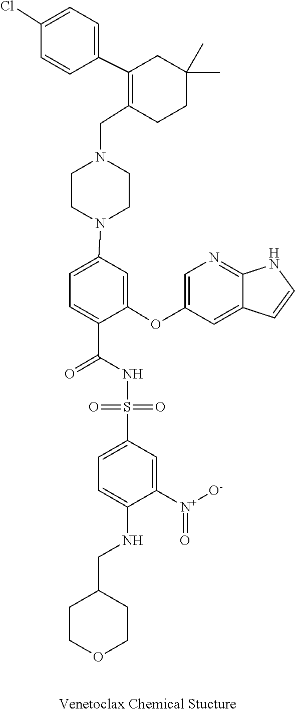 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(CCl)cc2)C1]

In a particular embodiment, the said pharmaceutical product is used in a method for treating unfit subjects, in particular older subjects.

In another particular embodiment, the said pharmaceutical product is used in a method for treating subjects likely to display an AML relapse and/or death, or subjects refractory or resistant to a first line treatment.

Another object of the invention is a compound targeting iron metabolism and disturbing intra-mitochondrial iron equilibrium, for use in the treatment of Acute Myloid Leukemia (AML).

The present invention will be now illustrated by the non-limitative examples.

### EXAMPLES

### Material and Methods

### Cell Culture

Human cell lines (Molm13, OCI-AML3 and MV4; 11) were maintained in RPMI-1640+ glutamine supplemented with 10% FCS, 100 IU ml-1penicillin, 100 ug ml-1 streptomycin under standard culture conditions (5% CO2, 37° C.). All cell lines were regularly tested and verified to be mycoplasma negative by PCR analysis by in-house genotyping. Human cell lines were authenticated by Short tandem repeat (STR) profiling through the Australian Genome Research Facility (Melbourne, Victoria).

### Cell Proliferation Assays and IC50 Determination

Cells were seeded at a constant density prior to treatment in triplicate and treated with either ironomycin or DMSO over the indicated time period. Drug was refreshed at least every two days. Cells were stained with DAPI and live cell number was calculated using the BD FACSVerse (BD Biosciences). CellTitreGlo (Promega) assays were performed as per manufacturer's instructions in a 96 well plate format. To determine the half-maximal inhibitory concentration (IC50) for ironomycin, 24 h after seeding the cells at a constant density in triplicate, cells were treated with ironomcyin or DMSO for 72 hours. At day 3, after incubating the cells with 600 μM of resazurin for 6 h, fluorescence was measured at λex=530 nm and λem=590 nm, using a Microplate Reader (EnSpire, Perkin Elmer). Relative fluorescence units were converted to per cent of inhibition relative to controls on the same plate, and the data were fitted against a four-parameter logistic model to determine the IC50.

### Compounds

Ironomycin (AM5) and AM23 were synthesized as previously described (Mai et al., 2017), venetoclax (ABT-199), biranapant and IDN-6556 are commercially available. Metformin and phenformin, were purchased from sigma (PHR1084 and PHR1573) and reconstituted freshly in culture medium. Turn-off Fe2+ probe (Petrat et al., 2002) was synthesized as previously reported (Muller et al., 2018), dose was 1 μM for 1 h for flow cytometry experiments. RhonoxM (Hirayarna et al., 2013) was synthesized as previously reported (Mai et al., 2017), dose was 1 μM, 1 h for flow cytometry experiments. Liproxstatin-1 (sigma SML1414, DMSO), ferrostatin (sigma SML0583, DMSO), RSL3 (sigma SML2234, DMSO), 6 aminonicotidamide (sigma A68203, DMSO), Z-VADfmk (R&D system FMK001, DMSO).

### Antibodies

ATF4 (cell signaling 11815), cleaved caspase 3, HK2 (abcam ab104836 1:500, milk), PARP1 (CST9542, 1:1000 BSA), PGP (Santa Cruz 390883, 1:250, milk), HSP60 are commercially available.

### Flow Cytometry

Cell death, cells were washed once with PBS and assessed using propidium iodide PI from staining (Sigma) according to manufacturer's instructions. Lipid peroxidation was assessed with BODIPYC11 staining (Thermo Fisher D3861). Cell were washed once with PBS and stained for 30 minutes in the dark at room temperature in 1 ml of 7.5 μM PBS solution, washed once and assessed according to manufacturer's instructions. Mitochondrial ROS were assessed with mitosox red probe (thermofisher M36008) according to manufacturer's instructions. All flow cytometry analyses were performed on a LSR Fortessa X-20 flow cytometer (BD Biosciences) and all data analysed with FlowJo. Cell sorting was performed on a FACSAria Fusion 5 (BD Biosciences).

### Western Blotting

Cells were lysed in 20 mM HEPES pIH7.9, 0.5 mM EDTA, 2% SDS plus 1× protease inhibitor cocktail (Roche) by brief sonication. Lysates were heated to 95° C. in SDS sample buffer with 50 mM DTT for 5 min, separated by SDS-PAGE and transferred to PVDF membrane (Millipore). Membranes were blocked in 5% milk or BSA in TBS +0.1% Tween-20, probed with the indicated antibodies, and reactive bands visualised using ECL Prime (GE).

### CRISPR sgRNA Library

The screen was performed using the Bassik Human CRISPR KO Library (Addgene 101296-101934). This 10-sgRNA-per-gene CRISPR/Cas9 deletion library was designed to target all ˜20,500 protein-coding human genes. The library contains two distinct classes of negative control gRNAs: non-targeting control sgRNA with no binding sites in the genome and safe-targeting sgRNA targeting genomic locations with no annotated function.

Further details are described in Morgens DW et al (Morgens et al., 2017). Library sgRNAs are expressed in the pMCB320 lentiviral sgRNA expression vector, which encodes puromycin and mCherry selection markers.

### CRISPR Screen

OCI-AML3 and MOLM13 cells were transduced with a lentiviral vector encoding Cas9 and selected with blasticidin. For the screen, 108 Cas9 cells were infected with the pooled lentiviral genome-wide sgRNA library at a multiplicity of infection of 0.3. The percent of cells infected was determined by flow-cytometry based evaluation of mCherry positive (sgRNA expressing) cells 72 hours (72 hr) following transduction. Infected cells were selected with 1 mg/mL puromycin for 72 hr, commencing 48 hr after transduction. Screen was performed in duplicate for each cell line with either DMSO or ironomycin 200 nM or 500 nM for OCI-AML3 or MOLM13, respectively, for 7 days and cultured for an additional 7 days in drug-free medium. Living cells (PI negative) were enriched by one round of FACS sorting at day 15 following transduction with the sgRNA library and a proportion of OCI-AML3 cells was retreated with 500 nM for 3 days following by another round of living cell FACS sorting. Genomic DNA was extracted (Puregene Core Kit A, Qiagen) from both the sorted cells and an unselected pool of mutagenized cells grown for the same amount of time. sgRNA sequences were amplified by two rounds of PCR, with the second-round primers containing adaptors for Illumina sequencing. Samples were sequenced with single-end 50 bp reads on an IIlumina HiSeq. The sequence reads were trimmed to remove the constant portion of the sgRNA sequences with fastx clipper (http://hannonlab.cshl.edu/fastx_toolkit/), then mapped to the reference sgRNA library with bowtie2. After filtering to remove multi-aligning reads, the read counts were computed for each sgRNA. The RSA algorithm (Konig et al., 2007) was used to rank the genes for which targeting sgRNA were significantly enriched in the sorted populations compared to the control unsorted populations grown in parallel.

### CRISPR/Cas9-Mediated Gene Disruption and Generation of Knockout Clones

Single guide RNA (sgRNA) oligonucleotides (Sigma-Aldrich) were cloned into lentiviral expression vector pKLVU6gRNA (Bbsl)-PGKpuro2ABFP as described (Addgene 50946). For CRISPR/Cas9 mediated gene disruption, cells were first transduced with the Cas9 expression vector pHRSIN-PSFFV-Cas9-PPGK-Blasticidin or FUCas9Cherry (Addgene 70182), and selected with blasticidin or sorted for mCherry expression respectively. To generate polyclonal populations with targeted gene disruption, cells were subsequently transduced with pKLVgRNA-PGKpuro2ABFP encoding either gene specific sgRNAs or with a control sgRNA targeting a ‘safe’ genomic location with no annotated function (Morgens et al., 2017). Efficient functional CRISPR/Cas9 mediated gene disruption of PGP and HK2 was confirmed by immunoblot.

### Metabolomics

MV4-11 cells were maintained in full growth medium and fresh medium was added at the time cells were treated with AM5. Following a 24-hour treatment, 3×106 MV4-11 cells were harvested by centrifugation, washed with normal saline and cell pellets were snap-frozen. For metabolite extraction, cell pellets were resuspended in 500 μL ice-cold MeOH:H20 (80:20) containing internal standards (13C-AMP, 13C-UMP, 13C Sorbitol, and 13C Valine) and vortexed. Samples were incubated on ice for 5 minutes, vortexed and debris was pelleted by centrifugation at 16,000 g for 10 minutes. The resulting supernatants were injected and analysed by hydrophilic interaction liquid chromatography (HILIC) and high-resolution mass spectrometry (Agilent 6545 LC/Q-TOF). Quality control checks were performed using QTOF MassHunter Quant software (Agilent) and metabolite peak calling was conducted using MAVEN analysis software (Agrawal et al., 2019)

### RNA Sequencing

RNA from cells was prepared using the Qiagen RNeasy kit. RNA concentration was quantified with a NanoDrop spectrophotometer (Thermo Scientific). Libraries were sequenced on the NextSeq500 using 75 bp single end chemistry.

### RNA Sequencing Analysis

Bcl2fastq (Illumina) was used to perform sample demultiplexing and to convert BCL files generated from the sequencing instrument into FastQ files. Reads were aligned to the human genome (G1k V37), or mouse genome (MM10) using HiSAT2 and reads were assigned to genes using htseq-count. Differential expression was calculated using either limma, voom or DESeq2. Genes with a false discovery rate corrected for multiple testing using the method of

Benjamini and Hochberg below 0.05 and a fold change greater than 1.5 were considered significantly differentially expressed. Heatmaps were generated in R using pheatmap.

### RT-QPCR, Primers

RNA was extracted using the Qiagen RNAeasy kit. cDNA was prepared using SuperScript VILO (Life Technolgies) according to manufacturer's instructions. Quantitative real-time PCR was performed on an Applied Biosystems StepOnePlus using Fast SYBR green reagents (Thermo Scientific). Expression levels were determined using the ΔΔCt method normalised to β2-microglobulin. DDIT3, CHAC1, CHOP and PSPH primers used for these studies have been published elsewhere (Quirós et al., 2017).

### Click Chemistry

Cells were treated with lysotracker deep red (Thermo Fisher L12492, 50 nM) directly in the culture medium for one hour, washed with culture medium once (400G, 4′) and resuspended in culture medium then treated for 1 hours with ironomycin 20 μM. After two PBS washes, 2.105 cells were cytospined (200G, 10′), fixed with 4% PFA PBS in pH 7.4 for 10 min at room temperature and washed three times with PBS. Cells were fixed with PBS containing 0.25% Tween-20 for 10min and wash in PBS-0.1% Tween three times for 5 min. The click reaction cocktail as shematized hereunder was prepared from Click-iT EdU Imaging kits (C10337, Life Technologies) according to the manufacturer's protocol, with slight modifications.

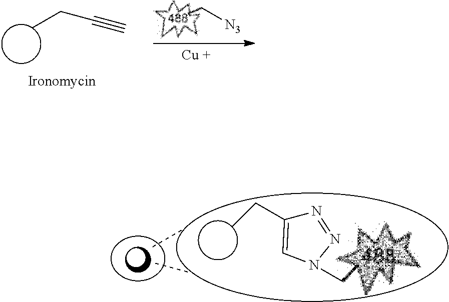

### Inductively Coupled Mass Spectrometry (ICP-MS)

Glass vials equipped with Teflon septa were cleaned with nitric acid 65% (VWR, Suprapur), washed with ultrapure water (Sigma-Aldrich) and dried. Cells were plated 24 h prior to the experiments. In all experiments, cells were incubated for 2 h with FBS-free medium prior to treatment. Cells were harvested using trypsinization (TrypLE Express Enzyme, Life Technologies) followed by two washes with 1× PBS. Cells were then counted using an automated cell counter (Entek) and transferred in 100 μL 1× PBS to the cleaned glass vials, and samples were lyophilized using a freeze dryer (CHRIST, 22080). Samples were subsequently mixed with nitric acid 65% overnight followed by heating at 80° C. for 2 h. Samples were diluted with ultrapure water (Sigma-Aldrich) to a final concentration of 0.475 N nitric acid and transferred to metal-free centrifuge vials (VWR, 89049-172) for subsequent ICP-MS analysis. ⁵⁶Fe concentrations were measured using an Agilent 7900 ICP-QMS in low-resolution mode. Sample introduction was achieved with a micro-nebulizer (MicroMist, 0.2 mL/min) through a Scott spray chamber. Isotopes were measured using a collision-reaction interface with helium gas (5 mL/min) to remove polyatomic interferences. Scandium and indium internal standards were injected after inline mixing with the samples to control the absence of signal drift and matrix effects. A mix of certified standards was measured at concentrations spanning those of the samples to convert count measurements to concentrations in the solution. Uncertainties on sample concentrations were calculated using algebraic propagation of ICP-MS blank and sample counts uncertainties. Values were normalized by dry weight and cell number.

### Transmission Electron Microscopy (TEM)

Cells were fixed in 2% GA in 0.1M sodium cacodylate buffer for 2 hours at room temperature, rinsed with 0.1M na-cacodylaatbuffer (5×10 min), and posffixation was done with 1% Osmium tetroxide and 1.5% Potassium ferricyanide for 2 hours at 4C in the dark. After rinsing in milliQ water, cells were stained with 0.5% Uranylacetate in MQ (±PH 4) for 1 h at 4° C. Cells were scraped and embedded in low melting agarose before serial dehydration in ethanol followed by propylene oxide and embedding into epon-araldyte resin. After polymerisation of the resin at 65 C for 48 hours, ultrathin sections were cut on a Leica UCT ultramicrotome and sections stained with uranyl acetate and lead citrate. Sections were examined on a Jeol JEM-1400Plus transmission electron microscope equipped with a Matataki flash camera at 80 keV.

### Primary AML Samples

Samples were collected from patients with AML treated at the Alfred Hospital after obtaining informed consent. Studies were conducted in accordance with the approved protocols through the Human Research Ethics Committee of the Alfred Hospital and the Declaration of Helsinki for all subjects. Primary AML cells were cultured in StemSpan SFEM (Stemcell Technologies) supplemented with 10 ng/mL IL-3, 10 ng/mL IL-6, 50 ng/mL SCF and 50 ng/mL FLT3L (all Peprotech) and 35 nM UM171 and 500 nM stemreginin (StemCell Technologies). For primary patient synergy analyses, 50,000 AML cells were plated in each well of a 96-well plate, and cultured with escalating doses of ironomycin and venetoclax for 5 days in medium conditions as described above. After 5 days, viability was assessed via propidium iodide staining and flow cytometry (BD FACSCanto). Synergy was determined using the Bliss synergy model.

### AML Xenograft Model

All mouse studies were conducted with approval from the Alfred Medical Research and Education Precinct Animal Ethics Committee. For in vivo studies, venetoclax was dissolved in 5% DMSO, 50% PEG 300, 5% Tween 80 and ddH2O 60%. Ironomycin was dissolved in PBS+2.5% DMSO. NOD-SCID IL2Rγ-null mice (NSG, 8-10 weeks old) were obtained from the Animal Resources Centre. MV4,11 cells (1.5×105) were transplanted into non-irradiated mice. Three days after transplantation, mice were treated with vehicle, or with ironomycin 1 mg/kg by IP (5 days/week for 4 weeks), venetoclax 75 mg/kg by oral gavage (5 days/week for 4 weeks) or the combination. A toxicity study was performed on non-irradiated NSG mice over 4 weeks with drug treatment as described above. Animals were euthanized at the completion of treatment. Body weight was assessed daily over 4 weeks and effects on the hematological system was analysed via full blood examination of peripheral blood (Cell-Dyn Hematology Analyser, Abbott) collected weekly.

### Synergy Experiments

A tecan D300e digital Dispenser automat was used to deliver drugs. Cell titer glow. Fluorescence was measured at λex 530 nm and λem 590 nm, using a Microplate Reader Cytation 5 (Biotek).

The softwares combenefit (Di Veroli et al., 2016) and compusyn (Zhang et al., 2016) were used to analyse the synergy between venetoclax and ironomycin.

Results:

Resistance Crispr Screen Identified PGP and HK2 as Two Major Regulators of Ironomycin Resistance

Ironomycin was tested in various human AML cell lines with various genetic backgrounds. Half maximal inhibitory concentration (IC50) of the drug varied from 14 nM to 410 nM at 72 hours as illustrated in the Table 1 hereunder:

TABLE 1

| AML CELL LINE | Driver mutation | IC50 (nM) | ATCC |
|---|---|---|---|
| KG1 | FGR1OP2-FGFR | 14 | CCL-246 |
| KASUMI-1 | AML1-ETO | 16 | CRL-2724 |
| HL-60 | NRAS | 26 | CCL-240 |
| NOMO-1 | MLL-AFF9 | 30 | ACC-542* |
| SKM-1 | EZH2-Y641C | 45 | ACC-547* |
| NB4 | PML-RARA | 47 | ACC-207* |
| MV4-11 | MLL-AF4 | 61 | CRL-9591 |
| MOLM-13 | M LL-AF9 | 70 | ACC-554* |
| OCI-AML3 | NPM1c | 118 | ACC-582* |
| HEL 92.1.7 | JAK2-V617F | 260 | TIB-180 |
| K 562 | BCR-ABL | 410 | CCL-243 |

*DSMZ

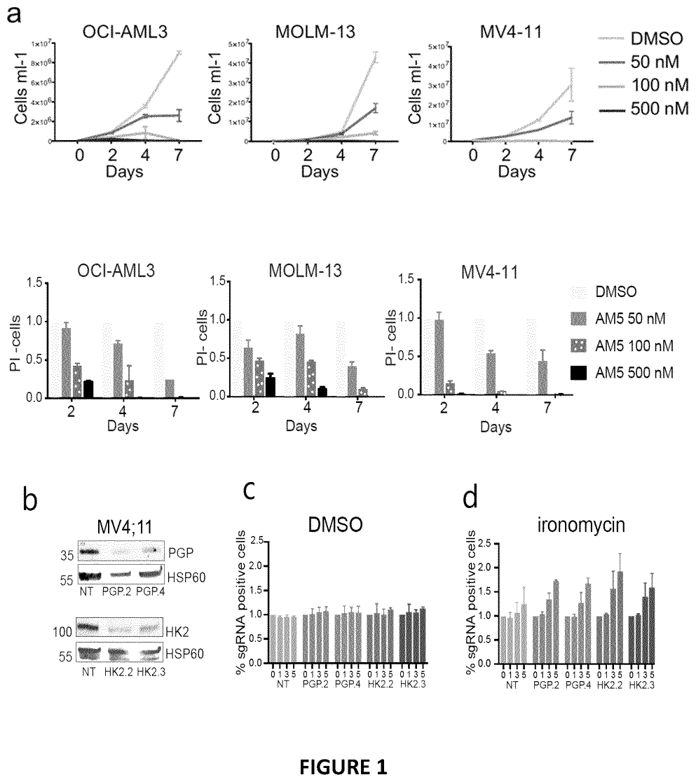

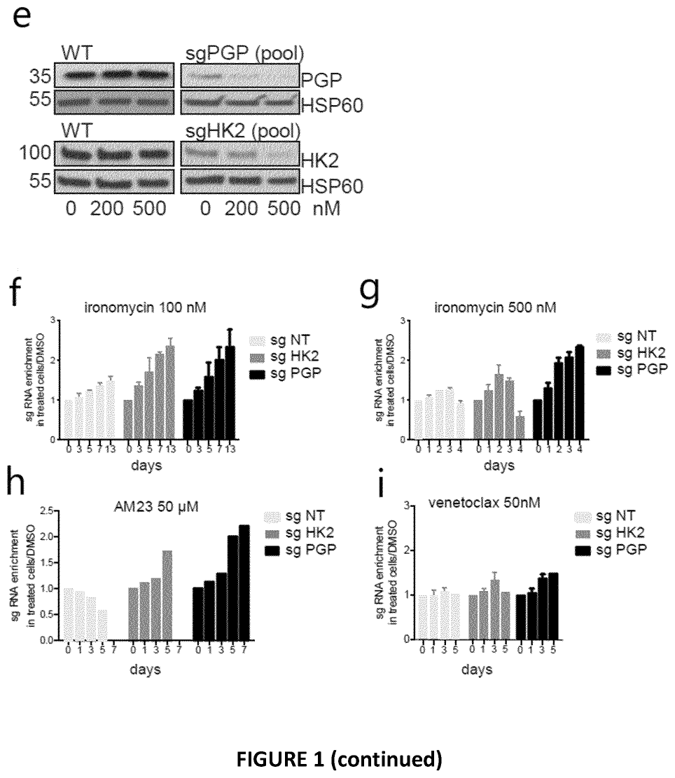

In all AML cell lines tested, ironomycin induced a proliferation block in a dose dependent manner (FIG. 1a top) and a rapid cell death as shown using PI staining (FIG. 1a bottom). To understand the molecular mechanisms underlying ironomycin activity, a whole genome CRISPR-cas9 resistance screen was performed on two sensitive AML cell lines with distinct genetic backgrounds, the MOLM13 and the OCI-AML3 cell lines. In both cell lines, phosphoglycerate phosphatase (PGP), a central phosphatase involved in glycolysis and pentose phosphate pathway (PPP) regulation were identified. Hexokinase 2 (HK2), the first enzyme of the glycolysis was also identified in the OCI-AML3 screen only , as well as Basigin (BSG), a multifunctional transmembrane glycoprotein linked to glucose and lactate cellular transport and several other genes involved in mitochondrial fission as Dynamin 1 Like (DNM1L) or tricarboxylic acid cycle (TCA) as the tricarboxylate transport protein SLC25A1.

KO cell lines both for HK2 and PGP genes were designed using two independent single sgRNAs (FIG. 1b) and a cell line with a pool of three independent sgRNAs (FIG. 1e). The results showed that ironomycin did not affect HK2 or PGP expression in the WT cell line (FIG. 1e). To validate the screen, a competition assays was performed mixing a 1:1 proportion of WT cells and KO cells. After ironomycin treatment, we found an enrichment in PGP KO and HK2 KO cells indicating a relative resistance as compared with the WT cell line (FIG. 1c-d). The survival advantage of the KO cell lines was independent of ironomycin doses (FIG. 1f-g) as well as specific, as we found a protective effect of the KO towards AM23, another salinomycin derivative ten times more potent than ironomycin (FIG. 1h). On the contrary, PGP and HK2 KO did not have any protective role towards the bcl2 inhibitor venetoclax (FIG. 1i). To conclude, loss of PGP and HK2 expression conferred ironomycin resistance.

### Metabolic Plasticity Impacted on Ironomycin Sensitivity

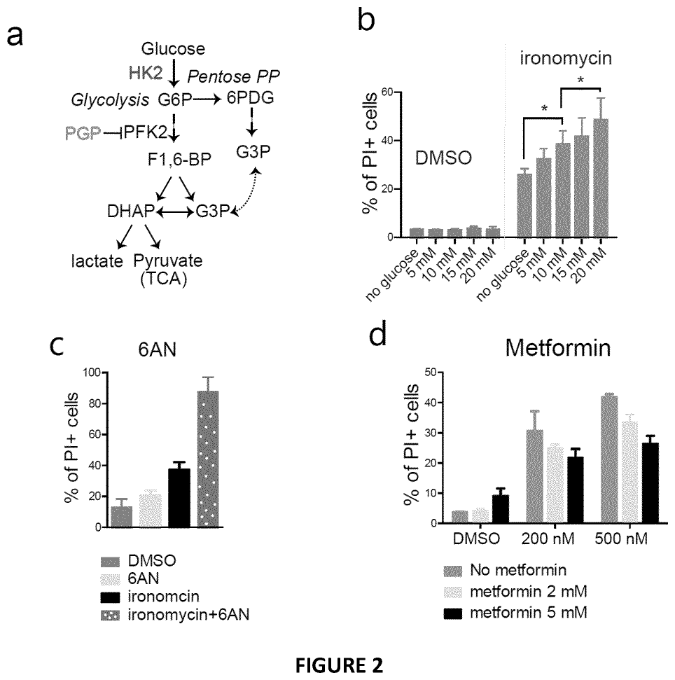

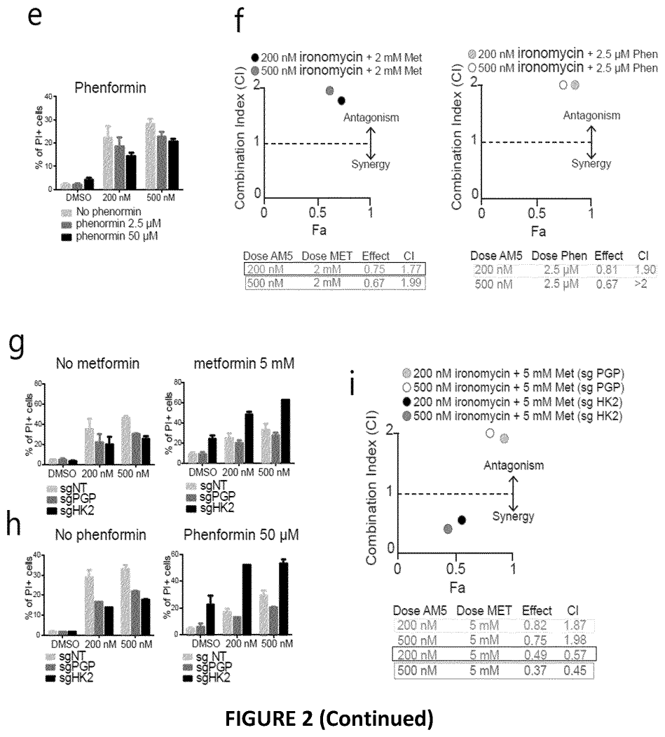

In order to identify metabolic profiles associated with ironomycin resistance a mass spectrometry metabolomics analysis of the untreated HK2 KO cell line compared to a nontargeting (NT) sgRNA control cell line was performed. HK2 is the first glycolytic enzyme phosphorylating glucose into Glucose-6-phosphate (G6P) (FIG. 2a). As expected, metabolites downstream of HK2 phosphorylation, including fructose-1,6-bisphosphate (F-1,6-BP) and dihydroxyacetone phosphate (DHAP), were decreased in the HK2 KO cell line. This result suggested that efficacy of the drug was dependent on intracellular glucose availability. In order to functionally validate this finding, the concentration of glucose was decreased in the culture medium and the results confirmed that ironomycin was less efficient in low glucose conditions. On the contrary, an increased glucose concentration resulted in a higher efficacy of the drug in killing AML cells (FIG. 2b).

The same experiment was performed with the PGP KO compared to NT cell line. PGP had been shown to be a central regulator of glycolysis through an indirect regulation of the key enzyme phosphofructokinase 2 (PFK2) (FIG. 2a). Consistently, metabolites downstream of PFK2 were downregulated whereas metabolites upstream of PFK2 were redirected towards the pentose phosphate pathway (PPP) as we observed that 6-phosphogluconate (6PG) as well as gluconate were up-regulated. These findings suggest that not only glucose concentration but also PPP is important in cell protection towards ironomycin. This finding was validated using the PPP inhibitor 6-aminonicotidamine (6AN) and we observed a strong toxicity of 6AN in association with ironomycin confirming that PPP protects against ironomycin (FIG. 2c). Given the role of PPP in detoxification against mitochondrial stress, we hypothesized that the drug was targeting mitochondrial cell respiration. This hypothesis was tested by pretreating AML cells with metformin or phenormin, two diabetes drugs from the biguanides class known to inhibit mitochondrial respiratory complex I. The results showed that biguanids rescued ironomycin cell death in every cell lines expect in the HK2 KO one (FIG. 2d-i). These findings were consistent with the fact that in HK2 KO cells, PPP was unable to provide any protection against ironomycin. Finally, in both KO cell lines, lactate was among the most upregulated metabolite (FIG. 2b), suggesting that aerobic glycolysis was indirectly upregulated in KO cells to compensate cellular respiration blockage. Collectively, these results indicated that metabolic plasticity of AML cell impacted on ironomycin sensitivity.

### Ironomycin Deeply Remodeled AML Cell Metabolism

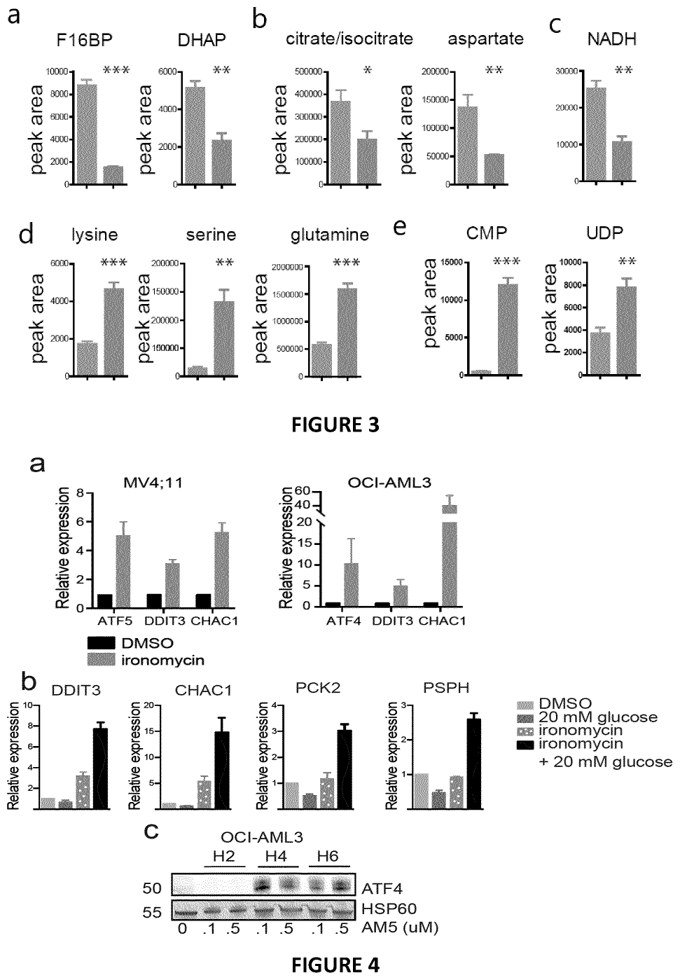

To explore the metabolic consequences induced by the drug, a mass spectrometry analysis of the metabolome was performed in the MV4; 11 cell lines as well as in the HK2 and PGP KO cell lines. In all the cell lines, ironomycin induced a profound downregulation of glycolytic metabolites such as fructose 1-6-bisphosphate (F1-6-BP), dihydroxyacetone phosphate (DHAP) and tricarboxylic acid cycle (TCA), citrate and aspartate as well as the reducing agent nicotidamide adenine dinucleotide (NADH), which mediates the transfer of electrons to the electron transfer chain to fuel ATP generation (FIG. 3a-c). In contrast, ironomycin increased aminoacids intracellular concentration, such as lysine, serine and glutamine, as well as nucleotidic acid metabolites such as cytidine monophosphate (CMP), uridine monophosphate (UMP) or guanosine monophosphate (GMP) (FIG. 3d-e). Together, metabolomics analyses suggested that ironomycin deeply remodeled AML cell metabolism in an attempt to cope with the cellular stress induced by ironomycin.

### Ironomycin Induces an ATF4 Dependent Stress Response

The transcriptional effect of the drug was assessed using RNA sequencing (RNAseq) analyses on MV4;11 treated with ironomycin alongside with the KO cell lines. No difference was observed between the WT and the KO cell lines, suggesting that ironomycin resistance was not mediated by transcriptomic profiles Among the upregulated genes, we found Activating Transcription Factor 4 (ATF4) and Activating Transcription Factor 5 (ATF5) and the major player of the unfolded protein stress response: DNA Damage Inducible Transcript 3 (DDIT3), also known as CCAAT/enhancer-binding protein Homologous protein (CHOP) plus several other genes involved in metabolism remodelling. This signature was recently described as a mitochondrial unfolded protein response (mtUPR), so-called “mitostress” signature and is a consequence of mitochondrial dysfunction (Quirós et al., 2017). Accordingly, GSEA analysis showed an enrichment in mitostress signature, whereas the whole set of genes from the Quirós et al. signature was up-regulated. In accordance with metabolomics data presented above, all the Kegg pathways upregulated were correlated with aminoacid metabolism. This was associated with an mRNA upregulation of the aminoacid transporters SLC38A2, SLC1A4, SLC38A1 and SLC7A11.

Finally, qPCR validation confirmed that ironomycin induced an early stress response involving ATF4, ATF5, DDIT3 and CHAC1 (FIG. 4a). Upregulation of the “mitostress” genes assessed by qPCR was even stronger in a high glucose culture condition (20 mmol), confirming that ironomycin takes advantage on glucose-dependent cellular respiration to induce the “mitostress” response (FIG. 4b). Finally, we confirmed that ATF4, the main transcription factor of the mitochondrial stress response (Quirós et al., 2017) was expressed at the protein level within 4-6 hours after ironomycin treatment (FIG. 4c). Together with metabolomics data shown above, RNAseq results suggested that the drug induces a mitochondrial stress response.

### Ironomycin Promotes Iron-Dependent Mitochondrial Oxidative Stress

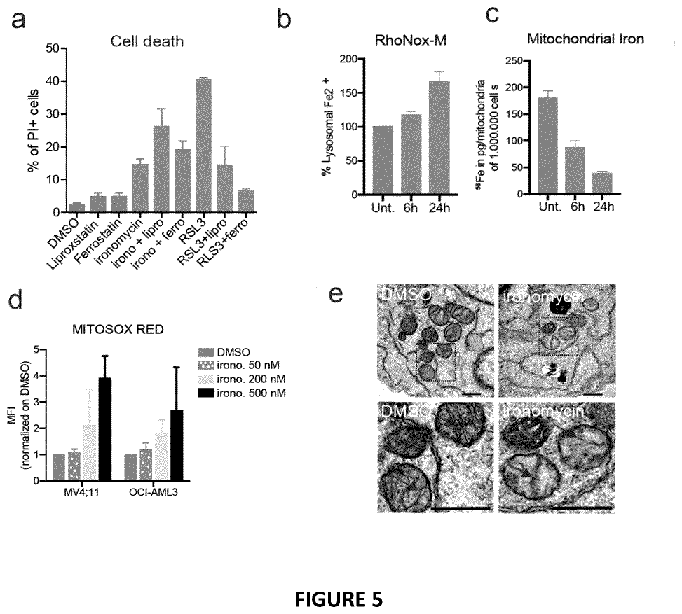

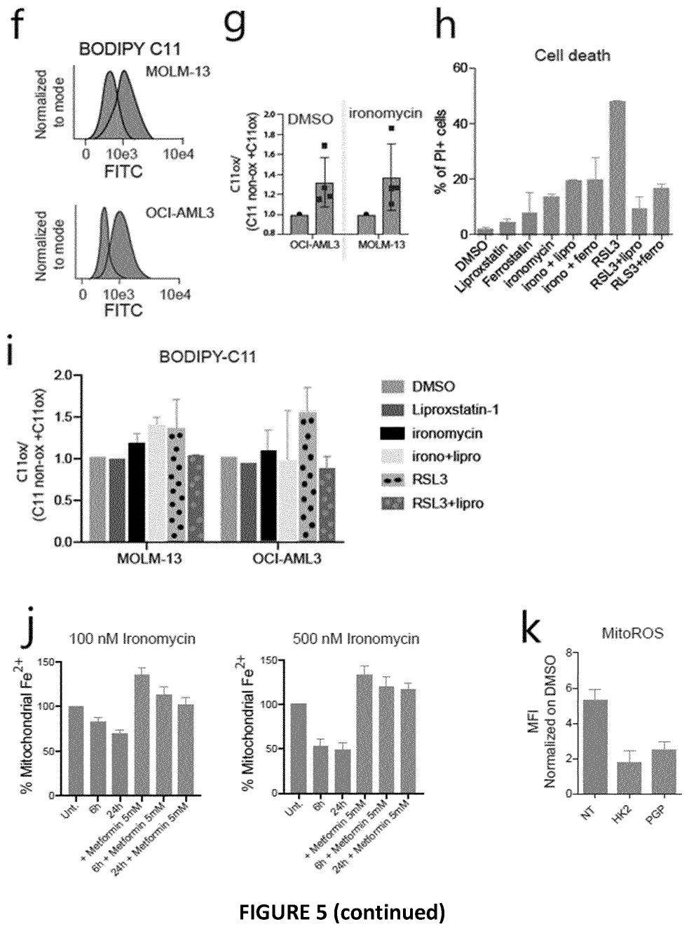

Canonical ferroptosis is a process in which iron induces iron dependent membrane lipid peroxidation through Fenton reaction and ROS generation, terminally leading to cell death (Dixon et al., 2012). We next asked whether ironomycin cell death was in fact canonical ferroptosis. We observed that the drug induced a lipid peroxidation as assessed with C11 BODIPY 581/591 staining. Nevertheless, this took place after 48 hours of treatment, suggesting that lipid peroxidation was not the primary event leading to cell death (FIG. 5f). Moreover, in contrast with findings regarding RSL3, a drug known to be a rapid ferroptotic inducer (Dixon et al., 2012), we failed to rescue lipid peroxidation nor cell death with the use of two ferroptosis inhibitors ferrostatin-1 and liproxstatin-1 (FIG. 5a & FIG. 5h). These results indicated that it is very unlikely that ironomycin is inducing canonical ferroptosis in our model.

Mitochondria are major hubs of iron utilization and accumulation. In particular, mitochondrial iron is a major component of electronic chain transport (ETC), responsible for mitochondrial respiration. Given the ability of ironomycin to sequester iron into lysosomes previously described (Mai et al., 2017), we next asked whether the drug was inducing intracellular iron imbalance in AML cells that could be responsible for the cell death.

First, taking advantage of the presence of a biorthogonal functional group in the ironomycin structure (Mai et al., 2017), we confirmed by click-chemistry that the drug spatially localizes with lysosomes in AML cells. Next, we confirmed that drug increased lysosomal iron, using RhoNoxM probe (FIG. 5b) (Niwa et al., 2014). Consistently, we observed a decrease in total iron into the mitochondria using Inductively Coupled Plasma-Mass spectrometry ICP-MS on isolated mitochondria after treatment (FIG. 5c). This suggested that the drug disturbed iron trafficking between the two organelles. By using a specific probe for mitochondrial iron, we found out that metformin counterbalanced mitochondrial iron content when combined with ironomycin, confirming that the two drugs were antagonistic (FIG. 5j). This was consistent with our prior results showing that biguanides rescued ironomycin-induced cell death (FIG. 2c-d & FIG. 2e-i). ETC being the major site of ROS production though OxPhos, we thus hypothesized that mitochondrial iron imbalance induced mitochondrial ROS. We found that the drug increased mitochondrial ROS after 24 hours of treatment (FIG. 5d). Mitochondrial ROS were found to be alleviated in the HK2 and PGP KO cell lines suggesting that a cellular context associated with a low cellular respiration was protective against oxidative stress (FIG. 5k).

Finally, using transmission electron microscopy (TEM) we observed that the drug disturbed the cristae structure. The cristae were disordered with abnormal spacing (FIG. 5e) suggesting a disorganization of the ETC. Together, these results suggest that the drug induced mitochondrial ROS and cristae disruption secondary to iron imbalance.

### Ironomycin Induces a Mitochondrial Non-Apoptotic Cell Death Synergistic with Venetoclax

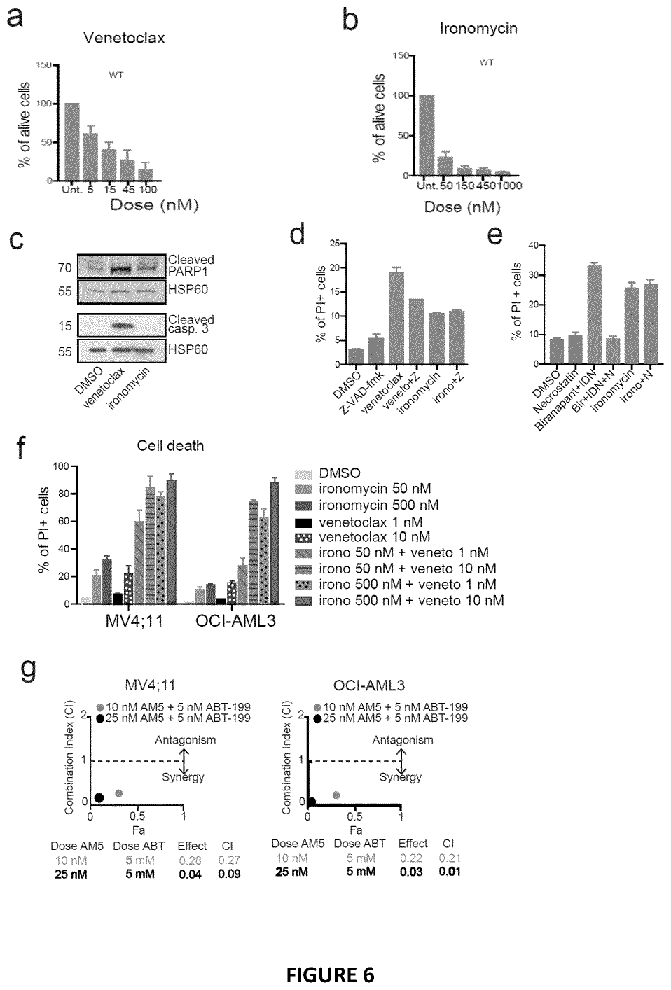

Finally, we asked whether the drug was inducing apoptosis. For this purpose, venetoclax, a drug known to induce AML cell apoptosis through Bcl2 inhibition (Pan et al., 2014), was used as a positive control. We found that both venetoclax and ironomycin induce cell death in WT MV4; 11 cell line (FIG. 6a-b). Contrary to venetoclax, ironomycin did not cleaved caspase 3 nor PARP1 (FIG. 6c) and the pan-caspase inhibitor Z-VAD-fmk did not rescue AM5-mediated cell death (FIG. 6d) arguing against canonical apoptosis. In addition, contrary to Birinapant+IDN-6556, a combination of drugs known to induce necroptosis in AML cells, cell death was not rescued by the necroptosis inhibitor necrostatin-1 (FIG. 6e). Collectively, these results suggested that ironomycin induced a non-canonical mitochondrial cell death.

We next combined venetoclax with ironomycin in order to synergize the efficiency of the two drugs, and demonstrated that the combination was highly effective in vitro in two cell lines (FIG. 6f). The synergy was confirmed with two mathematical models designed to assess synergy or antagonism between cytotoxic drugs (FIG. 6f-g) (Di Veroli et al., 2016; Zhang et al., 2016). Together, these results suggest that mitochondrial cell death induced by ironomycin synergizes with classical apoptosis raising the prospect that ironomycin may be used for patients with AML in association with venetoclax.

### Ironomycin Shows Marked Synergy with BH3 Mimetics and Overcomes Resistance to Venetoclax

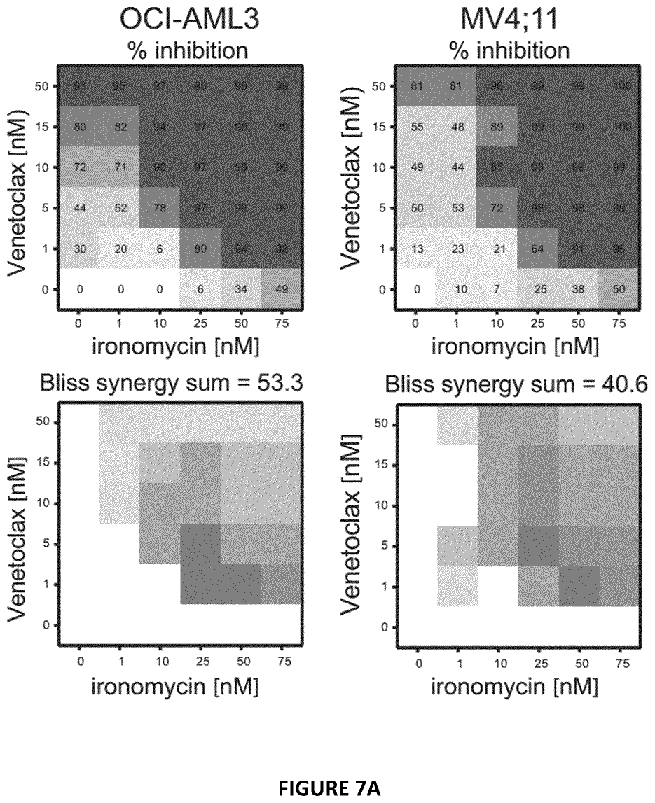

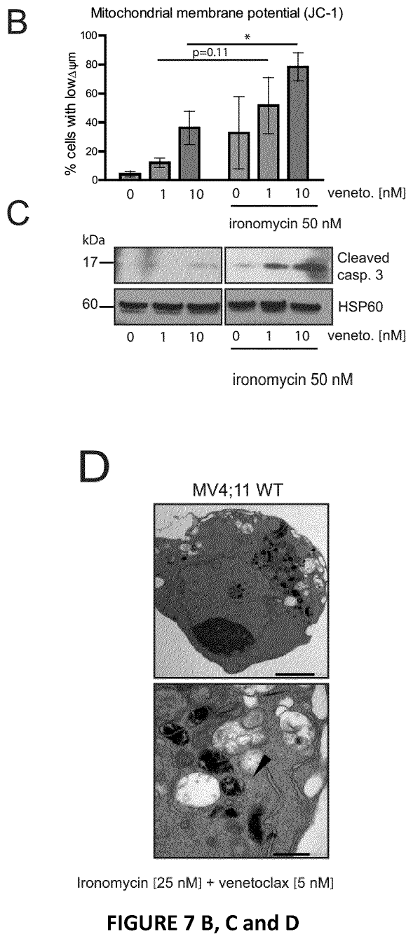

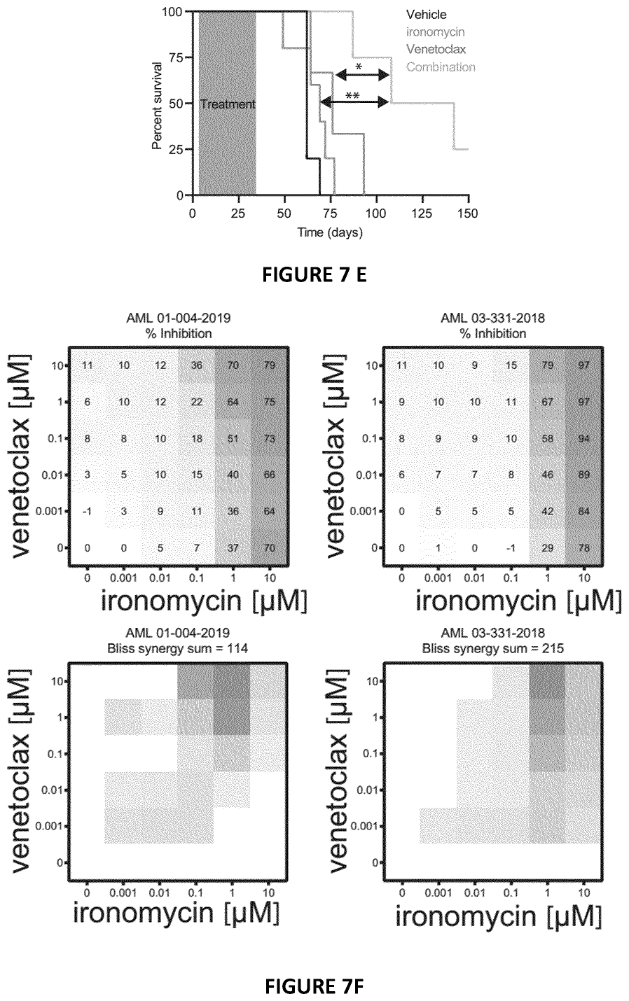

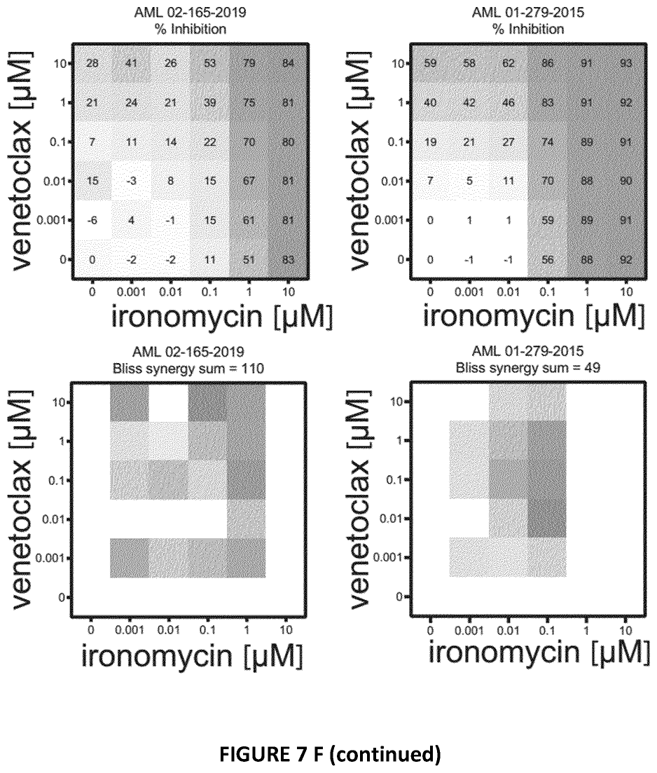

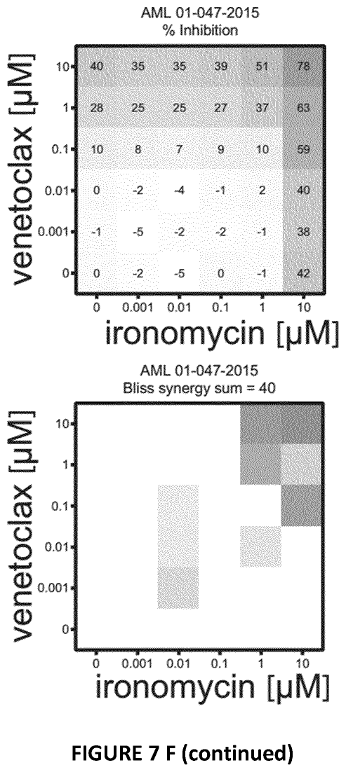

The striking activity of ironomycin in AML cells and the clear distinction in the mechanism of cell death to BH3 mimetics raised the possibility that ironomycin can dramatically enhance the activity of BH3 mimetics such as venetoclax. BH3 mimetics, such as venetoclax, have been paradigm-shifting through their clinical impact across a range of haematological malignancies, including AML where they have now been FDA approved (DiNardo et al., 2020). To assess the effects of ironomycin in combination with venetoclax, we first assessed the activity of the compounds in AML cell lines. These data showed dramatic synergy at low nanomolar concentrations of both drugs and Bliss synergy scores >40 in both cell lines (FIG. 7A).

Remarkably, in the presence of low doses of ironomycin even 1 nM of venetoclax is sufficient to result in a marked depolarization of mitochondrial membrane potential and the potent activation of caspases (FIG. 7B-C). When doses of both compounds, which have negligible effects in isolation, are combined there are dramatic cellular and mitochondrial morphological changes at an ultrastructural level that display features that are consistent with those seen when either venetoclax or ironomycin is used in isolation at much higher levels (FIG. 7D).

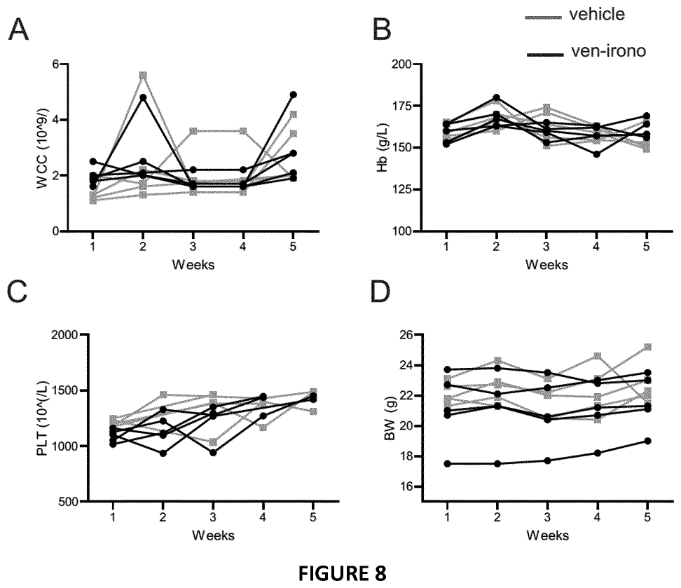

Although venetoclax and ironomycin have both been shown to have pre-clinical efficacy as single agents in pre-clinical models of cancer (Mai et al., 2017), the cornerstone of clinical cancer management is combination therapy. Here the major aspiration is to combine agents with non-overlapping mechanisms of action to enable the delivery of lower doses of the individual drugs, which in turn minimises side effects whilst maximising efficacy. To test the pre-clinical validity of combining ironomycin with venetoclax in vivo, we transplanted MV4,11 cells into NSG mice, a model which results in an aggressive and lethal malignancy. Following transplantation, mice were treated with a low dose of venetoclax and ironomycin alone or in combination. As expected, single agent low-dose therapy did not result in any survival advantage; however, the combination of venetoclax and ironomycin demonstrated significant efficacy with some of the mice surviving twice as long as mice treated with either single agents alone (FIG. 7E). Importantly, this highly effective combination therapy was also very well tolerated as there was no weight loss or discernible nadir in the blood counts of mice (FIG. 8A-D), suggesting no overt toxicity to normal haematopoiesis.

Venetoclax has unquestionably changed the natural history for many AML patients, with the vast majority of patients deriving a meaningful response to treatment. For subjects with primary or adaptive resistance to venetoclax-based therapy, median survival from treatment failure is only 2.4 months, highlighting the urgent and unmet need for more effective salvage options. To determine if ironomycin had activity in venetoclax resistant AML, five primary AML samples derived from patients with venetoclax resistant or refractory disease and varied genetic alterations (table 2 disclosed hereunder) were treated with venetoclax or ironomycin alone, or in chequerboard combination.

TABLE 2
Characteristics of clinical samples used for synergy experiment in FIG. 7F

| | | | | | | | Response to |
|---|---|---|---|---|---|---|---|
| | | | | Disease | Mutational | | venetoclax |
| AML ID | Sex | Age | AML type | status | profile | Karyotype | therapy |
| 01-004- | F | 78 | AML with | Primary | NRAS G13R | Normal | Refractory on |
| 2019 | | | MDS | refractory | SRSF2 P95L | | venetoclax |
| | | | related | | | | clinical trial |
| | | | changes | | | | |
| 03-331- | M | 22 | AML with | Relapsed | No mutations on | Complex | Relapsed on |
| 2018 | | | MDS | | a targeted panel | with | venetoclax + |
| | | | related | | | t(6;11) | azacitidine |
| | | | changes | | | | |
| 02-165- | F | 80 | AML with | De novo | SRSF2 P95L | Complex | Resistant to |
| 2019 | | | MDS | | | | venetoclax |
| | | | related | | | | ex vivo |
| | | | changes | | | | |
| 01-279- | F | 70 | AML | Relapsed | FLT3-ITD | Normal | Relapsed on |
| 2015 | | | without | | NPM1 W288fs | | venetoclax + |
| | | | maturation | | TET2 Q150* | | decitabine |
| | | | | | DNMT3A R882H | | |
| | | | | | FLT3 D835Y | | |
| 01-047- | M | 66 | AML with | De novo | FLT3-ITD | Normal | Refractory to |
| 2015 | | | minimal | | IDH1 R132C | | venetoclax + |
| | | | maturation | | RUNX1 A142D | | LDAC |
| | | | | | SRSF2 P95H | | |

Venetoclax impaired cell viability by 6-40% when administered as monotherapy for 5 days at a clinically relevant concentration of 1 μM (FIG. 7F). In contrast, combining the same concentration of venetoclax with 1 μM ironomycin enhanced the magnitude of cell death of these clinically resistant patient-derived samples to 51-91% over the same time interval, with the Bliss synergy sum confirming strong therapeutic synergy. These results demonstrate that the novel mechanism of ironomycin action can be leveraged to resensitize AML cells to venetoclax and substitute for cytotoxic drugs as a more effective therapeutic combination in the salvage setting.

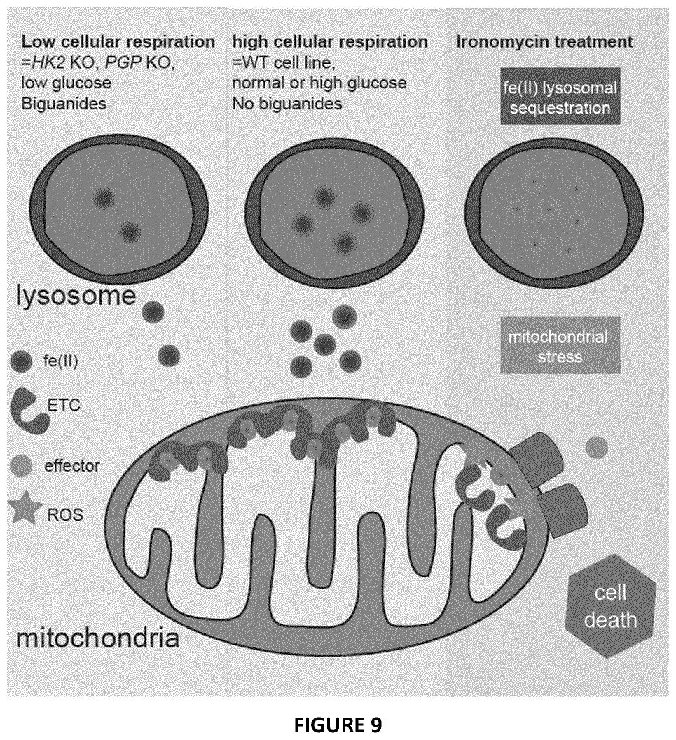

Schematic illustration of the ironomycin mechanism of cell death in AML is shown in FIG. 9.

### Discussion

In this study, an unbiased strategy was used to decipher ironomycin mechanism of action by running a whole genome resistance CRISPR screen. We identified genes related to mitochondria and glucose metabolism as the main regulators of ironomycin sensitivity. These unexpected findings opened the way to discover that the drug was a mitochondrial stress inducer, but also identified metabolic plasticity as a way to escape this oxidative stress. Knocking out HK2, a central enzyme regulating glycolysis, protected against ironomycin mitochondrial stress and subsequent cell death, confirming the previous findings that AML cells preferentially use glucose fuel for mitochondrial respiration (Pavlova and Thompson, 2016). Interestingly, knocking out PGP enzyme presented an additional advantages ; not only it decreased glucose dependent cell respiration but it also increased pentose phosphate pathway flux, consistently with the prior findings that PGP is a central regulator in cell detoxification. Indeed, PPP was found to provide the reducing equivalent NADPH and ultimately recycle oxidized glutathione that is strongly buffering mitochondrial stress

Combination of metabolomics and RNAseq revealed the ability of AML cells to develop alternative metabolic pathways to overcome mitochondrial stress. Metabolic remodeling was found to be coordinated by the mitochondrial unfolded protein response, also known as integrated stress response (ISR), mediated by the nuclear transcription factor ATF4 (Quirós et al., 2017). We believe that ironomycin, is inducing a mitochondrial stress by sequestering iron into lysosome, that consequently decreases mitochondrial iron and disturbs iron/sulfur clusters that are crucial component of mitochondrial ETC (Braymer and Lill, 2017).

Two recent studies identified that cytarabine-resistant AML (Farge et al., 2017) or poor prognosis AML patients (Baccelli et al., 2019) were mitochondria-dependent and could efficiently be targeted by mitochondrial drugs. Many groups developed therapeutic strategies inhibiting mitochondrial function, including drugs targeting the ETC (Baccelli et al., 2019), mitochondrial translation, mitochondrial DNA replication, Mitochondrial Protease CIpP and last but not least Bcl2 with venetoclax (Lagadinou et al., 2013; Pollyea et al., 2018). This last target is probably the most advanced as it is currently used in clinics (DiNardo et al., 2019). Cancer cells resistant to venetoclax developed sensitivity to other mitochondrial interruption as mitochondrial translation inhibition (Sharon et al., 2019) or mitochondrial structure modification (Chen et al., 2019). Our findings suggest that ironomycin could be used as a new drug targeting mitochondrion in an iron-dependent manner in AML but also, the synergy we observed between venetoclax and ironomycin led us to consider that a dual targeting of mitochondria could be a strategy to increase venetoclax efficiency in future clinical trials. We demonstrated in the present invention demonstrated that BH3 mimetics and ironomycin activate BAX/BAK through independent non-overlapping pathways. These findings have potential future clinical application as we find marked synergy with these drugs. Notably, combination with low doses of venetoclax and ironomycin are well tolerated in animals without any discernible systemic toxicity or nadir in peripheral blood counts. Yet at these doses the combination therapy results in marked anti-cancer activity resulting in an impressive survival advantage. Equally important is the fact that ironomycin overcomes resistance to venetoclax in patients who had failed clinical treatment with this agent. Together our data provide substantial pre-clinical evidence supporting the fact that ironomycin is a promising novel approach to negate the metabolic dependency on mitochondrial metabolism in cancers such as AML and induce mitochondrial outer membrane permeabilization (MOMP) mediated cell death in a synergistic manner to BH3 mimetics.

### REFERENCES

Agrawal S, Kumar S, Sehgal R, et al. El-MAVEN: A Fast, Robust, and User-Friendly Mass Spectrometry Data Processing Engine for Metabolomics. Methods Mol Biol. 2019; 1978:301-321. doi: 10.1007/978-1-4939-9236-2_19
Baccelli, I., Gareau, Y., Lehnertz, B., Gingras, S., Spinella, J.-F., Corneau, S., Mayotte, N., Girard, S., Frechette, M., Blouin-Chagnon, V., et al. (2019). Mubritinib Targets the Electron

Transport Chain Complex I and Reveals the Landscape of OXPHOS Dependency in Acute Myeloid Leukemia. Cancer Cell 36, 84-99.e8.
Braymer, J. J., and Lill, R. (2017). Iron-sulfur cluster biogenesis and trafficking in mitochondria. J. Biol. Chem. 292, 12754-12763.
Chen, X., Glytsou, C., Zhou, H., Narang, S., Reyna, D. E., Lopez, A., Sakellaropoulos, T., Gong, Y., Kloetgen, A., Yap, Y. S., et al. (2019). Targeting Mitochondrial Structure Sensitizes Acute Myeloid Leukemia to Venetoclax Treatment. Cancer Discov. 9, 890-909.
DiNardo, C. D., Pratz, K., Pullarkat, V., Jonas, B. A., Arellano, M., Becker, P. S., Frankfurt, O., Konopleva, M., Wei, A. H., Kantarjian, H. M., et al. (2019). Venetoclax combined with decitabine or azacitidine in treatment-naive, elderly patients with acute myeloid leukemia. Blood 133, 7-17.
DiNardo, C. D., Jonas, B. A., Pullarkat, V., Thirman, M. J., Garcia, J. S., Wei, A. H., Konopleva, M., Dohner, H., Letai, A., Fenaux, P., et al. (2020). Azacitidine and Venetoclax in Previously Untreated Acute Myeloid Leukemia. N Engl J Med 383,617-629
Di Veroli, G. Y., Fornari, C., Wang, D., Mollard, S., Bramhall, J. L., Richards, F. M., and Jodrell, D. I. (2016). Combenefit: an interactive platform for the analysis and visualization of drug combinations. Bioinforma. Oxf. Engl. 32,2866-2868.
Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.
Dobson, S. M., Garcia-Prat, L., Vanner, R. J., Wintersinger, J., Waanders, E., Gu, Z., McLeod, J., Gan, O. I., Grandal, I., Payne-Turner, D., et al. (2020). Relapse fated latent diagnosis subclones in acute B lineage leukaemia are drug tolerant and possess distinct metabolic programs. Cancer Discov.
Farge, T., Saland, E., de Toni, F., Aroua, N., Hosseini, M., Perry, R., Bosc, C., Sugita, M., Stuani, L., Fraisse, M., et al. (2017). Chemotherapy-Resistant Human Acute Myeloid Leukemia Cells Are Not Enriched for Leukemic Stem Cells but Require Oxidative Metabolism. Cancer Discov. 7,716-735.
Hirayama, T., Okuda, K., and Nagasawa, H. (2013). A highly selective turn-on fluorescent probe for iron(ii) to visualize labile iron in living cells. Chemical Science 4,1250-1256.
König R, Chiang C Y, Tu B P, et al. A probability-based approach for the analysis of large-scale RNAi screens. Nat Methods. 2007;4(10):847-849. doi:10.1038/nmeth1089
Lagadinou, E. D., Sach, A., Callahan, K., Rossi, R. M., Neering, S. J., Minhajuddin, M., Ashton, J. M., Pei, S., Grose, V., O'Dwyer, K. M., et al. (2013). BCL-2 inhibition targets oxidative phosphorylation and selectively eradicates quiescent human leukemia stem cells. Cell Stem Cell 12,329-341.
Mai, T. T., Hamaï, A., Hienzsch, A., Cañeque, T., Muller, S., Wicinski, J., Cabaud, O., Leroy, C., David, A., Acevedo, V., et al. (2017). Salinomycin kills cancer stem cells by sequestering iron in lysosomes. Nat. Chem. 9, 1025-1033.
Morgens, D., Wainberg, M., Boyle, E. et al. Genome-scale measurement of off-target activity using Cas9 toxicity in high-throughput screens. Nat Commun 8, 15178 (2017).
Muller, S., Versini, A., Sindikubwabo, F., Belthier, G., Niyomchon, S., Pannequin, J.,Grimaud, L., Caneque, T., and Rodriguez, R. (2018). Metformin reveals a mitochondrial copper addiction of mesenchymal cancer cells. PLoS One 13, e0206764.
Niwa, M., Hirayama, T., Okuda, K., and Nagasawa, H. (2014). A new class of high-contrast Fe(II) selective fluorescent probes based on spirocyclized scaffolds for visualization of intracellular labile iron delivered by transferrin. Org. Biomol. Chem. 12, 6590-6597.
Pan, R., Hogdal, L. J., Benito, J. M., Bucci, D., Han, L., Borthakur, G., Cortes, J., DeAngelo, D. J., Debose, L., Mu, H., et al. (2014). Selective BCL-2 inhibition by ABT-199 causes on-target cell death in acute myeloid leukemia. Cancer Discov. 4, 362-375.
Pavlova, N. N., and Thompson, C. B. (2016). The Emerging Hallmarks of Cancer Metabolism. Cell Metab. 23,27-47.
Petrat, F., Weisheit, D., Lensen, M., de Groot, H., Sustmann, R., and Rauen, U. (2002). Selective determination of mitochondrial chelatable iron in viable cells with a new fluorescent sensor. Biochem J 362,137-147.
Pollyea, D. A., Stevens, B. M., Jones, C. L., Winters, A., Pei, S., Minhajuddin, M., D'Alessandro, A., Culp-Hill, R., Riemondy, K. A., Gillen, A. E., et al. (2018). Venetoclax with azacitidine disrupts energy metabolism and targets leukemia stem cells in patients with acute myeloid leukemia. Nat. Med. 24, 1859-1866.
Quirós, P. M., Prado, M. A., Zamboni, N., D'Amico, D., Williams, R. W., Finley, D., Gygi, S. P., and Auwerx, J. (2017). Multi-omics analysis identifies ATF4 as a key regulator of the mitochondrial stress response in mammals. J. Cell Biol. 216, 2027-2045.
Sharon, D., Cathelin, S., Mirali, S., Di Trani, J. M., Yanofsky, D. J., Keon, K. A., Rubinstein, J. L., Schimmer, A. D., Ketela, T., and Chan, S. M. (2019). Inhibition of mitochondrial translation overcomes venetoclax resistance in AML through activation of the integrated stress response. Sci. Transl. Med. 11.
Wang, C., and Youle, R. J. (2009). The role of mitochondria in apoptosis*. Annu. Rev.

Genet. 43, 95-118.
Zhang, N., Fu, J.-N., and Chou, T.-C. (2016). Synergistic combination of microtubule targeting anticancer fludelone with cytoprotective panaxytriol derived from panax ginseng against MX-1 cells in vitro: experimental design and data analysis using the combination index method. Am. J. Cancer Res. 6, 97-104.

1-16. (canceled)

17. A method for treating subjects having Acute Myeloid Leukemia (AML), comprising administration of a pharmaceutical composition comprising, in a pharmaceutical acceptable vehicle, at least a compound of formula (I), enantiomers, mixture of enantiomers, diastereoisomers and mixture of diastereoisomers thereof: 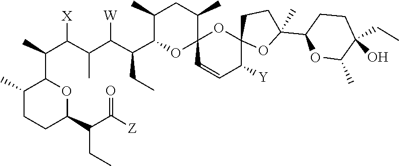
wherein:
W is selected from the group consisting of ═O; —NR₁R₂; —NR₃—(CH₂)ₙ—NR₄R₅;
O—(CH₂)ₙ—NR₄R₅; —NR₃—(CH₂)ₙ—N⁺R₆R₇R₈ and —O—(CH₂)ₙ—N⁺R₆R₇R₈;
X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl;
(C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl;
Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl;
(C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl;
(C₁-C₆)-alkyl-heteroaryl
n=0, 2, 3, 4, 5 or 6,
with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈.

18. The method according to claim 17, wherein the compound of formula (I) is with X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, (C₃-C₁₆)-cycloalkyl, (C₁-C₆)-alkyl-aryl and (C₁-C₆)-alkyl-heteroaryl.

19. The method according to claim 17, wherein the compound of formula (I) is with X is OH, Z is OH and Y is NR₁R₂ where R₁ is H and R₂ is selected from the group consisting of (C₈-C₁₄)-alkyl; (C₃-C₅)-alkenyl; (C₃-C₅)-alkynyl, (C₃-C₆)-cycloalkyl, benzyl, and CH₂-pyridynyl.

20. The method according to claim 17, wherein the compound of formula (I) is with W is ═O, X is OH, Z is OH, and Y is NR₁R₂ where R₁ is H and R₂ is selected from the group consisting of (C₃-C₅)-alkynyl and (C₃-C₆)-cycloalkyl.

21. The method according to claim 20, wherein the compound of formula (I) is with W is ═O, X is OH, Z is OH, and Y is NR₁R₂ where R₁ is H and R₂ is a (C₃-C₆)-cycloalkyl group.

22. The method according to claim 20, wherein the compound of formula (I) is with W is ═O, X is OH, Z is OH, and Y is NR₁R₂ where R₁ is H and R₂ is a (C₃-C₅)-alkynyl group.

23. The method according to claim 17, wherein the subjects having Acute Myeloid Leukemia (AML) are unfit subjects or older subjects.

24. The method according to claim 17, wherein the subjects having Acute Myeloid Leukemia (AML) are likely to display an AML relapse and/or death, or subjects refractory or resistant to a first line treatment.

25. The method according to claim 17, wherein the pharmaceutical composition is a pharmaceutical combination product comprising (ii) another anti-cancer agent selected from the group consisting of agents used in chemotherapy, targeted treatments, immune therapies, and combinations thereof, wherein administration of (i) the compound of formula (I) and (ii) the other anti-cancer agent is simultaneous, separate, or staggered.

26. A pharmaceutical product comprising:
(i) a compound of formula (I) according to claim 17 wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in targeted treatments selected from Bcl2 inhibitor, Mcl1 inhibitor or other BH3 mimetics, FLT3 inhibitors, IDH1/IDH2 inhibitors, and combinations thereof.

27. The pharmaceutical product according to claim 26, wherein the agent (ii) used in targeted treatments is selected from Bcl-2 inhibitor, other BH3 mimetic, and a combination thereof.

28. The pharmaceutical product according to claim 26 further comprising (iii) an additional agent used in chemotherapy.

29. The pharmaceutical product according to claim 28, wherein the (iii) additional agent used in chemotherapy is selected from anthracyclin, aracytine, and azacitidine.

30. The pharmaceutical product according to claim 26 comprising:
(i) the compound of formula (I) with W is ═O, X is OH, Z is OH, and Y is NR₁R₂ where R₁ is H and R₂ is selected from the group consisting of (C₃-C₅)-alkynyl and (C₃-C₆)-cycloalkyl, and
(ii) BH3 mimetic.

31. The pharmaceutical product according to claim 30, wherein
(i) the compound of formula (I) with W is ═O, X is OH, Z is OH, and Y is NR₁R₂ where R₁ is H and R₂ is (C₃-C₅)-alkynyl and
(ii) BH3 mimetic is the compound ABT-199 (Venetoclax)

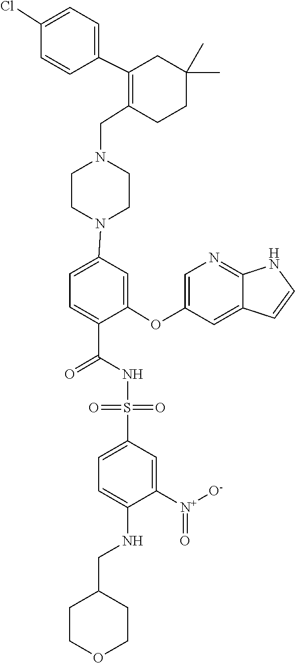 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(CCl)cc2)C1]

32. The method according to claim 23, comprising administration of a pharmaceutical product comprising:
(i) a compound of formula (I) 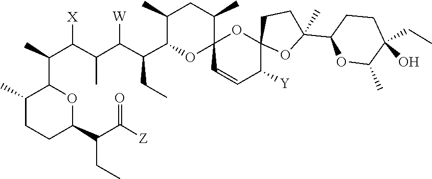
wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in targeted treatments selected from Bcl2 inhibitor, Mcl1 inhibitor or other BH3 mimetics, FLT3 inhibitors, IDH1/IDH2 inhibitors, and combinations thereof.

33. The method according to claim 24, comprising administration of a pharmaceutical product comprising:
(i) a compound of formula (I) 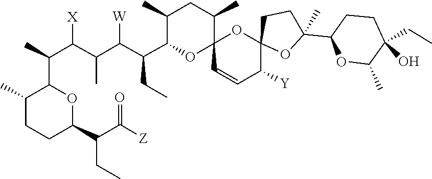
wherein W is ═O, X is OH, Z is OH and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl, and
(ii) an agent used in targeted treatments selected from Bcl2 inhibitor, Mcl1 inhibitor or other BH3 mimetics, FLT3 inhibitors, IDH1/IDH2 inhibitors, and combinations thereof.

34. A method for treating subjects having Acute Myeloid Leukemia (AML), comprising administration of a pharmaceutical composition comprising, in a pharmaceutical acceptable vehicle, at least a compound targeting iron metabolism and disturbing intra-mitochondrial iron equilibrium.
